(19) United States
(12) Patent Application Publication
(10) Pub. No.: US 2022/0042030 A1
(43) Pub. Date: Feb. 10, 2022

(54) A METHOD TO IMPROVE THE AGRONOMIC CHARACTERISTICS OF PLANTS

(71) Applicants: UNIVERSIDAD NACIONAL DEL LITORAL, Santa Fe (AR); CONSEJO NACIONAL DE INVESTIGACIONES CIENTIFICAS Y TECNICAS (CONICET), Ciudad Autonoma de Buenos Aires (AR)

(72) Inventors: Renata REINHEIMER, Santa Fe (AR); Carolina BELLINO, Santa Fe (AR)

(21) Appl. No.: 17/440,647

(22) PCT Filed: Mar. 18, 2020
(86) PCT No.: PCT/IB2020/052483

§ 371 Date: Sep. 17, 2021

(30) Foreign Application Priority Data
Mar. 19, 2019 (AR) 20190100693

(51) Int. Cl. C12N 15/82

(52) U.S. Cl. CPC C12N 15/8262

(57) ABSTRACT

A method to improve the agronomic characteristics of plants by genetically transforming the plant with a nucleic acid sequence encoding RAMOSA1 transcription factor, where the plant is selected from the group consisting of gramineous monocotyledons of the BOP clade, monocotyledons-non-grasses and dicotyledons. In addition, sequences of DNA, cDNA, protein, cells, uses, genetic transformation method of plants and transformed plants are described.

### FIELD OF TECHNOLOGY

The present invention belongs to the field of biotechnology and improvement of plant species of agronomic interest

### STATE OF THE ART

The grass family (Poaceae or informally called “grasses”) encompasses approximately 11,000 species among which there are prominent cereals such as corn, rice, oats, wheat, barley, rye, sugarcane, among others, which constitute a very important part of human intakes (Grass Phylogeny Working Group II, 2012). Grass species are distributed in 13 subfamilies, three of them evolved early (Anomochloideae, Pharoideae and Puelioideae) and the rest are grouped into two large lineages: the BOP clade and the PACMAD clade (Soreng et al., 2015). Inside the clade BOP are grouped the commonly called winter cereals such as rice, wheat and oats. On the other hand, within the PACMAD summer cereals are found as corn, sorghum and sugarcane.

Grasses are morphologically unique among Angiosperms (plants with flowers), since they are characterized by highly modified flowers, grouped together in structures called spikelets (Cheng et al., 1983; Clifford 1987; Ikeda et al., 2004). Spikelets are distributed on different systems of inflorescences branch, which configures a variety of reproductive structures truly remarkable and novel.

The morphology of grass inflorescence is known to be extremely variable among species, complex in its development, in addition to being genetically and agronomically important (Malcomber et al., 2006). The final morphology of an inflorescence of grasses determines the production of seeds (grains) and is dictated, mainly, by the activity of axillary meristems which may be undetermined (with the production of branches) or determined (with the formation of spikelets). Understand the molecular mechanism that controls where and when the change from undetermined to determined axillary meristem occurs is key when designing projects that aim to modify the final form of the inflorescence of a species to increase the yield of a crop.

Recently a molecular mechanism was described through which axillary meristems can acquire the determined fate in the maize inflorescence (Gallavotti et al., 2010). In this model, RAMOSA1 (RA1) and RAMOSA1 ENHANCER LOCUS2 (REL2) form a complex, interacting via the EAR (Ethylene-responsive element binding factor-associated amphiphilic repression) motives of RA1 and the CTLH domain (C-terminal to lyssencephaly typel-like homology, LISH) of REL2. The RA1/REL2 complex binds to the promoter of a target gene (probably LIGULELESS1 (LG1), Eveland et al., 2014) to promote the determined fate in the axillary meristems of the inflorescence. In this model, RAMOSA3 (RA3), which codes for the enzyme trehalose-6-phosphatase (TPP), and RA2, which codes for a LATERAL ORGAN BOUNDARY Domain (LOB) protein, regulate RA1 transcript levels upstream (Bortiri et al., 2006; Satoh-Nagasawa et al., 2006).

RAMOSA1 is a transcription factor of the Cys2-His2 zinc finger type (C2H2). The first zinc finger protein discovered was TFIIIA isolated from Xenopus leavis (Hanas et al., 1983). Since then, zinc finger proteins were isolated and characterized from prokaryotic and eukaryotic organisms (Takatsuji et al., 1998). The origin of the zinc finger domain is controversial, mainly because it widely varies e in structure (Krishna et al., 2003; Malgieri et al., 2015). The diversity of the zinc finger domain implies a wide range of functions from DNA and RNA binding up to protein-protein interaction and association with membranes (Laity et al., 2001; Krishna et al., 2003). The term “zinc finger”, of animals and plants, describes a nucleic acid binding domain in a protein that folds around a Zinc ion coordinated in tetrahedra (Miller et al., 1985; Isernia et al., 2003; Brayer et al., 2008).

The amino acids that coordinate the Zinc ion are always cysteine or histidine residues, however, there is diversity in the sequence and length of the zinc finger domain. Zinc finger proteins may contain different domains of the same or different type of zinc finger. In nature there is an additional variability due to the association of zinc finger domains with other domains. For example, some zinc finger proteins are associated with ring finger domains or spiral-spiral domains, to form a domain called tripartite. There are different types of zinc fingers, such as C2H2, C2HC, C2C2. Type C2H2 is known as the classic zinc finger domain and comprises the majority of zinc finger proteins constituting one of the largest families of transcription factors of the eukaryotic domain (Tupler et al., 2001; Brayer et al., 2008). Normally, there are two criteria used to classify proteins with zinc fingers, the first being the type of zinc finger and the second the number of zinc fingers present in the protein. For example, zinc finger proteins of the C2H2 type may contain from one to 40 zinc finger domains (Englbretch et al., 2004; Brayer et al., 2008). Zinc finger proteins with a single C2H2 domain have been characterized in plants, for example, SUPERMAN (SUP) from Arabidopsis and RAMOSA1 (RA1) from maize (Sakai et al., 1995; Vollbrecht et al., 2005).

The zinc finger motif C2H2 (ZF-C2H2) is the classic zinc finger domain. It was first recognized in Xenopus transcription factor IIIA (TFIIIA) (Miller et al., 1985). The domain is typically 25 to 30 amino acids in length. The following pattern describes the zinc finger*XCX (1-5) —C—X3—*X5—*X2—HX (3-6)—*[H/C], where X can be any amino acid, and the numbers in brackets indicate the number of residues. The positions marked with * are those that are important for the stable folding of the zinc finger. The final position can be either His or Cys, while remaining a C2H2 zinc finger domain. In view of recent publications on the design of zinc finger domains it is also feasible to replace one or more of the Cys or His amino acids, while still retaining the original functionality of the C2H2 domain. The residues that separate the second Cys and the first His are mainly polar and basic. The canonical zinc finger is composed of two short beta chains followed by an alpha helix. The DNA binding of the zinc finger motif is mediated by an amino terminal part of the alpha helix that joins the major groove in the zinc fingers for DNA binding. C2H2 domains have been shown to interact with RNA, DNA, and proteins. The tetracoordination of a Zinc ion by the conserved cysteine and histidine residues determines the conserved tertiary structure of the motif. The conserved hydrophobic residues are commonly found at positions -2 and also 4 amino acids after the second cysteine (which participates in the Zinc bond) and in position three before the first histidine (which participates in the binding of Zinc).

The zinc finger of plants is characterized by a highly conserved sequence of six amino acids, located within a surface that makes contact with putative DNA of each finger. Two forms of such conserved sequences are most found in the C2H2 zinc fingers of a plant, the QALGGH and the NNM/WQMH. Despite the high conservation of the QALGGH sequence, certain variants occur in nature, more typically +1 “Q” can be a “G”, “K” or “R” (these amino acids share the same characteristic at the same time), the +2 “A” can be “S” (which share the same characteristic of small amino acids) or the +3 “L” can be “F” (these two amino acids are both hydrophobic). In the NNM/WQMH motif in position 3 there is mainly an “M” or a “W”.

RAMOSA1 from maize, is a transcription factor formed by 175 amino acids (525 base pairs) (Vollbrecht et al., 2005). The protein is composed of a single C2H2 zinc finger domain that binds to DNA through a short a-helix that contains the amino acid sequence QGLGGH, with a glycine residue that relaxes the helix (Vollbrecht et al., 2005). Downstream of the zinc finger, two EAR repressor domains (LxLxLxL) have previously been identified (Vollbrecht et al., 2005). The EAR domain is a amphiphilic repression motif associated with ERF. Ohta et al. (2001) characterized the EAR motifs present in zinc finger proteins. The characteristic pattern of the EAR motif is the conserved sequence hDLNh (X) P, where h is a hydrophobic residue (any of A, C, F, G, H, I, K, L, M, R, T, N, W , Y), more typically L/F/I, and where “X” may be one (any amino acid) or no amino acid. A characteristic feature of the EAR motive is the alternation of hydrophilic and hydrophobic residues being the residue of aspartic acid (D) amphiphilic.

RAMOSA1 is a transcription factor that localized in the cell nucleus. The transport of RAMOSA1 to the cell nucleus is done by the presence of a nuclear localization signal. Traditionally, this nuclear localization signal consists of a group of basic amino acids that resembles the B box (basic box) described by Takatsuji et al. (1992). This type of box has been recognized in proteins that carry one or more zinc fingers (Sakamoto et al., 2000). The group is rich in Lysine (K) and Arginine (R) residues. A consensus sequence that defines the most frequent form of B box for C2H2 genes is KR (S) KRXR, where “S” in the third position may be absent or present. However, other variants may occur in nature that still retain the characteristic of being a charged region rich in basic amino acids. The location of the basic box is more frequently in the N-terminal of the protein, but it can also occur elsewhere. It has been speculated that due to its basic nature, B box could also participate in DNA binding. However, in the amino acid sequence of RAMOSA1 there is no traditional nuclear localization signal located in the N-terminal, as has been described for other zinc finger proteins. In contrast, in RAMOSA1 the nuclear localization signal is located in the QGLGGH motif where “L” and “H” seem to have an important role in the importation of this protein into the cell nucleus (Yang, 2011).

It is interesting to note that RA1 is a locus that was selected during the process of maize domestication (Sigmon and Vollbrecht, 2010). However, its evolution throughout the Angiosperms is still unclear. In terms of sequence similarity, RA1 is similar to SUPERMAN (SUP) of Arabidopsis (Sakai et al., 1995; Vollbrecht et al., 2005). Within the functional context, SUP intervenes during floral development avoiding the initiation of supernumerary stamens, while RA1 plays a central role in the development of inflorescence and does not appear to control floral development (Sakai et al., 1995; Vollbrecht et al., 2005). Overexpression of RA1 (35S::RA1, Cassani et al., 2006) in Arabidopsis sup5 mutants fail to restore the number of stamens in the flower. Also, Arabidopsis 35S::RA1 transgenic plants generate pleiotropic effects in the plant as an increase in the size of the reproductive organs due to cell expansion (Landoni et al., 2007). These results indicate that indeed the role of RA1 of maize functionally differs from that of Arabidopsis.

On the other hand, the international patent application WO0190343 describes the RA1 gene and the RAMOSA1 protein from maize, where in addition to isolating the sequence the effects of suppressing the gene by transposons such as the Mutant Suppressor (Spm) are described. The Argentine patent AR042679, describes a method to modify the agronomic characteristics of plants, and the nucleic acids to modify it. The products of the expression of these nucleic acids are zinc finger proteins, with two zinc fingers of type C2H2 (2×C2H2), each zinc finger of sequence QALGGH, with NNM/WQMH motifs, 1 EAR motif. This protein is used to transform rice plants generating a 68% increase in seed production and also showing an increase in the biomass of the plant.

The present invention solves the problem of generating transgenic plants with improved agronomic characteristics, such as an increase in biomass, an increase in seed production and in the size of roots and converting into perennials the plants that originally were not.

### BRIEF DESCRIPTION OF FIGURES

FIG. 1. Comparison of the peptide sequences of RAMOSA1 of maize (Zea mays) and its homologue in Setaria viridis and Cenchrus equinatus, with the domains: zinc finger (solid line), towards the N terminal, shared by all sequences; two EAR domains, (double line), towards terminal C, shared by all sequences; and an EAR domain (dotted double line), close to the zinc finger, only in RA1 of maize.

FIG. 2. Schematic representation of the constructions generated and used in the present invention. The constructions corresponding to the destination vector with the coding sequences of RAMOSA1 are shown. (A) Schematic representation of the construction corresponding to the destination vector with the RAMOSA1 coding sequences of Zea mays. (B) Schematic representation of the construction corresponding to the destination vector with the coding sequences of RAMOSA1 of Setaria viridis. (C) Schematic representation of the constructions corresponding to the destination vector with the coding sequences of RAMOSA1 of Cenchrus equinatus. (D) Schematic representation of the constructions corresponding to the destination vector with the coding sequences of RAMOSA1 of Zea mays used for rice transformation.

FIG. 3. The homozygous transgenic lines used in the different experiments with relatively low (G1), intermediate (G3) and high (G2) levels of transgene expression. (A) Photograph showing the phenotype of transgenic lines with level G1, G2 and G3 of transgene expression. (B) Number of leaves of the G1, G2 and G3 transgenic plants up to 45 days post-emergence. (C) Height (measured in centimeters) of the G1, G2 and G3 transgenic plants until 50 days post-emergence. (D) Quantification of the relative expression levels of G1, G2 and G3 transgenic lines. (E) Arabidopsis thaliana wild type plant. (F) Two months old Arabidopsis transgenic plant overexpressing CeRA1 showing similar phenotype than plants overexpressing SvRA1.

FIG. 4. Photograph documenting the phenotype of two transgenic plant lines (L4-G2 and L6-G2) in the middle of the life cycle in comparison with a wild type plant at the end of the life cycle.

FIG. 5. Photograph showing the phenotype of the transgenic line L4-G2 at the end of the life cycle.

FIG. 6. Height of transgenic plants in relation to wild plants. (A) values of maximum height expressed in millimeters (mm) and difference in height between the transgenic lines in relation to wild control plants expressed in numbers of times. (B) graph that documents the values in (A).

FIG. 7. Covered area above the ground of transgenic plants in comparison to wild plants. (A) Maximum coverage values expressed in square millimeters (mm²) and difference in covered area above ground between the transgenic lines in comparison to wild control plants expressed in numbers of times. (B) graph that documents the values in A. (C) Example of photography used in the measurements of area above ground.

FIG. 8. Development of the root system of transgenic plants compared to wild control plants. (A) Photograph documenting the phenotype of transgenic plant roots compared to a wild plant at 5 days post-germination. (B) Photograph documenting the phenotype of transgenic plant roots compared to a wild type plant at 10 days post-germination.

FIG. 9. Increase in root biomass by modifying parameters of root development. (A) Length (in mm) of the main root of wild plants (gray) and Ubi::SvRA1 (black), in the 15 days after germination. Each point represents the average value and its error obtained from an N=6. (B) Total length (in mm) of the main root and lateral roots of wild plants (gray) and Ubi::SvRA1 (black), in the 15 days after germination. Each point represents the average value and its error obtained from an N=6. (C) Total area (in mm) occupied by the roots of wild plants (gray) and Ubi::SvRA1 (black), in the 15 days after germination. Each point represents the average value and its error obtained from an N=6. (D) Number of lateral roots of wild plants (gray) and Ubi::SvRA1 (black), in the 15 days after germination. Each point represents the average value and its error obtained from an N=6.

FIG. 10. Development of the root system in the presence of biotic factors of transgenic plants compared to wild control plants. Photograph documenting the phenotype of transgenic plant roots compared to a wild plant at 10 days post-germination.

FIG. 11. Increase in root biomass by modifying parameters of root development in the presence of biotic stress. (A) Length (in mm) of the main root of wild plants (gray) and Ubi::SvRA1 (black), in the 15 days after germination. Each point represents the average value and its error obtained from an N=4. (B) Total length (in mm) of the main root and lateral roots of wild plants (gray) and Ubi::SvRA1 (black), in the 15 days after germination. Each point represents the average value and its error obtained from an N=4. (C) Total area (in mm) occupied by the wild plants (gray) and Ubi::SvRA1 (black), in the 15 days after germination. Each point represents the average value and its error obtained from an N=4. (D) Number of lateral roots of wild plants (gray) and Ubi::SvRA1 (black), in the 15 days after germination. Each point represents the average value and its error obtained from an N=4.

FIG. 12. Comparison of Ubi::ZmRA1 plants with wild plants. (A) 37 day post germination plants. (B) Height of wild plants (black) and Ubi::ZmRA1 (gray), in the 45 days after germination. Each point represents the average value and its error obtained from an N=4. The data analysis was performed with the statistical variable t-student (p-value <0.05). (C) Number of leaves of wild plants (black) and Ubi::ZmRA1 (gray), in the 45 days after germination. Each point represents the average value and its error obtained from an N=4. Data analysis was performed with the t-student statistical variable (p-value <0.05).

FIG. 13. Ubi::ZmRA1 plant silique. (A) Silique. (B) Open silique, releasing its seeds (58), and formed by three carpels *. (C) graphic documenting the number of seeds per silique between transgenic lines and wild control plants.

FIG. 14. Phenotype of transgenic rice plant expressing Ubi:ZmRA1 compared to control plants. Example of rice transgenic lines with relatively low levels, intermediates, and high levels of expression of construct Ubi::ZmRA1.

FIG. 15. Plant height phenotype of transgenic rice plant expressing Ubi::ZmRA1 compared to control plants. (A) Example of rice transgenic lines with semi-dwarf phenotype. (B) Graphic documenting the height (cm) of the transgenic plants (black bars) in comparison with control plant (grey bars).

FIG. 16. Number of reproductive tillers phenotype of transgenic rice plant expressing Ubi::ZmRA1 compared to control plants and the correlation with semi-dwarf phenotype. (A) Example of rice transgenic lines with semi-dwarf phenotype. (B) Graphic documenting the number of reproductive tiller of the transgenic plants (black bars) in comparison with control plant (grey bars). (C) Graphic documenting the correlation of number of reproductive tiller (black bars) versus plant height of the transgenic plants in comparison with control plants (grey bars).

### BRIEF DESCRIPTION

The present invention describes a method for improving the agronomic characteristics of a plant which comprises genetically transforming the plant with a nucleic acid sequence that encodes the RAMOSA1 transcription factor, where the plant is selected from the set comprised of grass- monocotyledons of the BOP clade, nongrass-monocotyledonous and dicotyledonous. Preferably, the nucleic acid sequence encodes RAMOSA1 transcription factor from PACMAD clade. More referentially, the nucleic acid sequence encodes RAMOSA1 transcription factor from plants of the genus Setaria, Cenchrus or Zea; wherein said nucleic acid sequence encoding for RAMOSA1 transcription factor from Setaria viridis, Zea mays or Cenchrus equinatus are selected from the group: SEQ ID. Not, SEQ ID. No. 2, and SEQ ID. No 3.

In another embodiment of the present invention, said nucleic acid encoding RAMOSA1 is overexpressed with a plant or seed promoter.

The process of the present invention improves the agronomic characteristics of a plant since: it increases at least 30%, preferably at least 50% seed production, at least doubles biomass, extends at least 100% the life of the transformed plant or produces a combination of at least two of these improvements.

To achieve the agronomic improvement, another object of the present invention is an isolated DNA sequence comprising at least 90% homology to SEQ IDs. No1, SEQ ID No2 or SEQ ID No3. Preferably said nucleic acid sequence comprises at least 95% homology to SEQ ID. No1, to SEQ ID No2 or to SEQ ID No3. More preferably, it comprises at least 98% homology to SEQ ID. No1, or SEQ ID No2 or to SEQ ID No3. Even more preferably, it comprises at least 99% homology to SEQ ID. No1 or to SEQ ID No2, or to SEQ ID No3.

In another embodiment of the present invention, said isolated DNA sequence is cDNA.

In addition to the DNA or cDNA sequences, it comprises a promoter for overexpression selected from the set comprising: promoters of the actin, ubiquitin, pEMU, MAS, corn histone H4, rice, Panicum virgatum, Setaria; peanut chlorotic caulimovirus (PCISV) promoter; 35S promoter of cauliflower mosaic virus (CaMV); the complete promoter of tabacco mosaic virus (FMV); the ALSO gene promoter from Brassica napus; various promoters of Agrobacterium genes; and own promoters of Setaria viridis, Cenchrus equinatus and Zea mays.

Another object of the present invention is an isolated protein that comprises at least 90% homology to SEQ IDs. No4, SEQ ID No5, or SEQ ID No6; preferably it comprises at least 95% homology with SEQ IDs. No4, SEQ ID No5, or SEQ ID No6; more preferably it comprises at least 99% homology with SEQ IDs. No4, SEQ ID No5, or SEQ ID No6.

Another object of the present invention is the use of the nucleic acid of SEQ ID. No1, or SEQ ID. No2 or SEQ ID No3 to increase biomass, root growth, seed production and the life of a plant that includes the introduction of these sequences in the plant.

Another object of the present invention is a genetic construct comprising at least one expression control sequence, a nucleic acid to be expressed and optionally, a transcription termination sequence, characterized in that the nucleic acid to be expressed encodes the transcription factor RAMOSA1. Where said nucleic acid to be expressed is selected from the group comprised by the sequences: SEQ ID. No1, SEQ ID. No2 and No3.

In an alternative embodiment of the present invention, the genetic construct is a vector selected from the group comprised by pANIC and pCAMBIA.

Another object of the present invention is a genetically modified cell characterized in that it comprises a nucleic acid sequence encoding for the RAMOSA1 transcription factor. Wherein said nucleic acid sequence encoding for the RAMOSA1 transcription factor is selected from the group consisting of: SEQ ID. No1, SEQ ID. No2, and SEQ ID. No3. Wherein said genetically modified cell is selected from the group consisting of: prokaryotic cell, insect cell, animal cell and plant cell; preferably, said genetically modified cell is selected from the group consisting of: Escherichia coli and Agrobacterium tumefaciens.

In addition, another object of the present invention is a method for obtaining transgenic plants with improved agronomic characteristics compared to wild plants, where said characteristics are selected from the group comprising: increased biomass, increased root growth, increased seed production and increased plant life; where the method includes:
introducing into a plant or a plant cell a nucleic acid of sequence selected from the group consisting of: SEQ ID No. 1, SEQ ID No. 2 and SEQ ID No. 3.
cultivate the plant or plant cell under conditions that promote its growth.

Another embodiment of the present invention is a method for obtaining transgenic plants with improved agronomic characteristics compared to wild plants, wherein said improved agronomic characteristics are selected from the group consisting of: increasing their biomass by at least 30%, preferably at least 50%, 50% seed production, and extends the life of said plant by 100%, the method comprising:
introducing into a plant or a plant cell a nucleic acid of sequence selected from the group consisting of: SEQ ID. No. 1, SEQ ID No. 2 and SEQ ID No. 3;
cultivate the plant or plant cell under conditions that promote its growth.

The present invention also describes a grass-monocotiledoneous, non-grass monocotyledonous or dicotyledonous transgenic plant characterized in that it comprises the nucleic acid sequence encoding for RAMOSA1 transcription factor. Where said nucleic acid sequence is selected from the group consisting of: SEQ ID. No1, SEQ ID. No2 and SEQ ID No. 3. Where, in addition, said transgenic plant, in comparison with the native ones, has increased its biomass, its seed production and its life cycle.

### DETAIL DESCRIPTION OF THE INVENTION

The present invention describes a method to improve the agronomic characteristics of a plant. The agronomic characteristics that are improved by the present invention are selected from the set comprising: increase in biomass, increase in root growth, increase in seed production, increase in life cycle.

The method of the present invention applicable to a type of plants that are characterized by not containing the RA1 gene and therefore, do not possess the RAMOSA1 transcription factor. It can be generalized that these plants are BOP clade grass monocots, non-grass monocots and dicots. It can also be said that the present procedure is applicable to plants that do not belong to the PACMAD clade.

In a preferred embodiment of the present invention, the procedure described here consists in genetically transforming a plant that does not originally possess the RAMOSA1 gene and therefore does not possess the RAMOSA1 transcription factor. The genetic transformation comprises introducing a nucleic acid encoding for RAMOSA1 to the plant by transgenesis. The nucleic acid encoding for RAMOSA1 can be obtained from any of the plants belonging to the PACMAD clade, more preferably from species of the Panicoideae, Aristidoideae, Chloridoideae, Micrairoideae, Arundinoideae, Danthoinioideae subfamilies. For example, but without restricting the protection spectrum of the present invention, the nucleic acid encoding for RAMOSA1 can be obtained from plants of the Setaria, Cenchrus or Zea genus, specifically from Setaria viridis, Cenchrus equinatus and Zea mays.

The term “transformation” as used herein encompasses the transfer of an exogenous polynucleotide into a host cell, regardless of the method used for the transfer. Plant tissue capable of subsequent clonal propagation, either by organogenesis or embryogenesis, can be transformed with a genetic construct of the present invention and regenerate an entire plant from there. The particular tissue chosen will vary depending on the clonal propagation systems available, and more suitable, for the particular species that is being transformed. Examples of target tissues include leaf discs, pollen, embryos, cotyledons, hypocotyledons, megagametophytes, callus tissue, existing meristematic tissue (eg, cotyledon meristem, hypocotyledon meristem). The nucleic acid can be transiently or stably introduced into a host cell and can be maintained in a non-integrated manner, for example as a plasmid. Alternatively, it can be integrated into the host genome.

The transformation of a plant species is currently a routine technique. Advantageously, any of the different transformation methods can be used to introduce the nucleic acid of interest (for example, the nucleic acid encoding for RAMOSA1 transcription factor) into a suitable progenitor cell. Transformation methods include the use of liposomes, electroporation, chemicals that increase the absorption of free DNA, injection of DNA directly into the plant, particle gun bombardment, transformation using virus or pollen, and microprojection. The methods can be selected from the calcium/polyethylene glycol method for protoplasts; protoplast electroporation; microinjection into plant material; bombardment of particles coated with DNA or RNA; infection with viruses (non-integrators) and the like. A preferred transformation method is an Agrobacterium-mediated transformation method.

Obtaining the nucleic acid encoding RAMOSA1 transcription factor can be carried out by any of the methods widely known in the state of the art. In general, a screening is carried out in search of the DNA sequence of the RAMOSA1 gene, and the necessary oligonucleotides are made to carry out the amplification, cloning in vectors and subsequent transformation of cells and/or plants.

Typically, after transformation, plant cells or groups of cells are selected for the presence of one or more markers that are encoded by the genes that can be expressed by the plant transferred in conjunction with the gene of interest, after which regenerates the transformed material into an entire plant.

After DNA transfer and regeneration, the presumed transformed plants can be evaluated, for example, using a Southern analysis, to detect the presence of the gene of interest, the number of copies and/or the genomic organization, alternative or additionally, the expression levels of the recently introduced DNA can be measured by means of Northern and/or Western analysis, quantitative polymerase chain reaction, both techniques being well known to those skilled in the art.

The transformed plants generated can be propagated by a wide variety of means, such as by clonal propagation, or by classical plant breeding techniques. For example, a first generation (or T1) of transformed plants can be self-pollinate to produce a second generation of homozygous (or T2) transformants, and T2 plants further propagated through classical plant breeding techniques.

The generated transformed organisms can take a variety of forms. For example, they can be chimeras of transformed cells and non-transformed cells; clonal transformants (eg, all cells transformed to contain the expression cassette); transformed and untransformed tissue grafts (eg, in plants, a transformed rhizome grafted to an untransformed stem).

The present invention extends to any plant or plant cell produced by any of the methods described herein, and to all parts of the plant and propagules thereof. The present invention is further extended to encompass the progeny of a first transfected or transfected cell, tissue, organ, or plant that has been produced by any of the aforementioned methods, the only requirement being that the progeny exhibit the same genotypic and/or phenotypic characteristics as those produced in the parents by means of methods such as those described here.

The invention also describes genetically modified host cells that comprise a nucleic acid encoding RAMOSA1 transcription factor. Such preferred host cells as described herein are derived from a plant, algae, bacteria, fungus, yeast, insect, or animal. The invention also encompasses harvestable parts of a plant, such as, but not limited to, seeds, leaves, fruits, flowers, petals, stamens, mother crops, stems, rhizomes, roots, tubers, bulbs, or cotton fibers.

In the present invention, it is described which plants comprise the nucleic acid encoding RAMOSA1 transcription factor, its isolation is also described for its subsequent use. In a preferred form, but without limiting the spectrum of protection of the invention, the isolation and use of the nucleic acid encoding RAMOSA1 of Setaria viridis, Cenchrus equinatus and Zea mays is shown in the examples of the present invention.

The present invention further describes genetic constructs and vectors to facilitate the introduction and/or to facilitate expression of the nucleic acid sequences of the present invention, wherein said genetic construct and vectors comprise: (i) a nucleic acid capable of modifying expression of a nucleic acid encoding RAMOSA1 transcription factor; (ii) one or more control sequences capable of directing the expression of said nucleic acid sequence encoding RAMOSA1; and optionally, (iii) a transcription termination sequence. The genetic constructs and vectors are widely known in the state of the art, being able to be made by recombinant DNA technology and, in addition, they can be inserted into commercially available vectors. In a preferred form, the expression vectors to be used in the present invention are plant expression vectors. In another preferred form, the cloning vector comprises a promoter sequence for sequence overexpression in plants or seeds, preferably monocotyledonous plants. More preferably, but not limited to, the vector is selected from the set comprised of pANIC vectors and pCAMBIA vectors, more preferably pANIC6A (Mann et al., 2012). The vector further comprises a cassette for overexpression of the nucleic acid encoding RAMOSA1 transcription factor. Overexpression caused by a strong promoter, the use of transcription enhancers or translation enhancers. The term overexpression as used herein means any form of expression that is additional to the level of the original wild-type expression. Preferably, the nucleic acid that is introduced into the plant and/or the nucleic acid that is overexpressed in the plant is in the sense direction with respect to the promoter with which it is operatively linked. Promoters that can be used to overexpress the nucleic acid encoding RAMOSA1 are selected from the set comprised of, but not limited to: promoters for the actin, ubiquitin, pEMU, MAS, histone H4 genes from maize, rice, Panicum virgatum, Setaria; peanut chlorotic caulimovirus (PCISV) promoter; 35S promoter of cauliflower mosaic virus (CaMV); the complete promoter of tabacco mosaic virus (FMV); the ALSO gene promoter from Brassica napus; various promoters of Agrobacterium genes; and tissue-specific promoters such as the SvRA1 and ZmRA1 self-promoter.

The present invention further describes transgenic plants with modified agronomic characteristics. The agronomic characteristics are any of the group consisting of: increased biomass, increased root growth, increased seed production, increased life cycle. The transgenic plants have been genetically transformed with a nucleic acid sequence encoding RAMOSA1 protein that gives the plants the modified agronomic characteristics. Preferably said nucleic acid is DNA. More preferably, said nucleic acid es cDNA.

Furthermore, the present invention describes a method for obtaining transgenic plants with improved agronomic characteristics. Where the method comprises introducing a nucleic acid encoding RAMOSA1 transcription factor into BOP clade grass-monocotyledonous plants, dicotyledonous and non-grass monocotyledonous plants, or into plant cells of BOP clade grass-monocotyledonous, non-grass manocotyledoneous and dicotyledoneaous; and subsequently cultivate the plant or plant cell under favorable conditions for its growth.

### EXAMPLES

1) Reconstruction of the Molecular Evolution of RAMOSA1 (RA1) and Identification of Homologs.

To date, the information available on the evolution of RA1 in Angiosperms is poor. RA1 is known to be a transcription factor that is present in maize and its closest relatives within the tribe Andropogoneae (members of the PACMAD clade) including sugarcane, sorghum, but is absent in the rice and Brachypodium distachyon, two members of the BOP lineage (Reinheimer and Kellogg, unpublished data).

In order to identify the origin of RA1, understand its evolution in grasses, and identify homologs, we reconstructed a phylogenetic tree with sequences of the coding region obtained from BLAST searches in genomes of grasses and other Angiosperms (Musa acuminata, Ananas comosus, Arabidopsis thaliana, Aquilegia coerulea, Cucumis sativus, Medicago truncatula, Carica papaya, Populus trichocarpa, Solanum lycopersicum and Ricinus comunis) deposited in Phytozome v.12 (https://phytozome.jgi.doe.gov/pz/portal.html) using the Cys2 region-His2 of the SUP finger zinc domain and RA1. The zinc finger domain, of approximately 30 amino acids, of all the obtained sequences was aligned using the MAFFT software (Katoh et al., 2002). From this alignment, the molecular evolution of all the obtained zinc finger sequences was reconstructed following the methodology explained below. As a result of this analysis we obtained a tree divided into two large lineages. One of these lineages is made up of grass sequences (including RA1) sister to a clade consisting of grass-monocot and non-grass monocots and dicot sequences, including SUP, RABBIT EARS (RBE), ZINC-FINGER PROTEIN 10 and 11 (ZFP10, ZFP11) from Arabidopsis. From these results a new data set was generated with the sequences that exclusively belongs to this lineage. The complete sequences of this new matrix were converted to peptide sequences and manually aligned in MEGA v.6.06 (Tamura et al., 2013) based on the functional motifs identified by the Motif-based sequence analysis tool software (Bailey et al., 2009). Then, the aligned matrix was converted to nucleotide sequences for further analysis. Trees were reconstructed using the Monte Carlo Markov Chain algorithmic method implemented in MrBayes v.3.1.2 (Huelsenbeck and Ronquist, 2001) and the GTR+G+I model inferred in MrModeltest v.2.3 (Nylander, 2004) based on the Akaike criterion. Two independent chains were run for 30 million generations and trees were sampled every 1000 generations. The analysis was repeated twice, starting with random trees. The convergence and the effective sample size for each replicate was verified using Tracer v.1.5 software (Rambaut and Drummond, 2007). Finally, a majority rule consensus tree (45,002 trees) was reconstructed after discarding the trees of the first 7.5 million generations (25%). The sequences of the genes coding for ZFP10, ZFP11, RBE and SUP proteins were used as reference and outgroup sequences.

In order to detect additional motifs in the RA1 peptide sequence and its homologs, a data set was constructed with the complete peptide sequences of RA1 and its identified homologs. The data set was scanned with the Motif-based sequence analysis tool software (Bailey et al., 2009) available in the MEME v4.12 interface. The searches were performed using up to 15 domains between 6 to 40 amino acids long and default parameters. Only the motifs with E values less than 1e-50 were considered.

RESULTS: The tree topology obtained indicates that RAMOSA1 is an exclusive protein of monocotyledonous plants, of the Order Poales, of the Family Poaceae (grasses), of the clade traditionally known by the name of PACMAD (Soreng et al., 2015) that includes the Panicoideae, Aristidoideae, Chloridoideae, Micrairoideae, Arundinoideae, Danthoinioideae subfamilies.

From the searches carried out on the genomes of monocotyledonous-non-grasses (Musa acuminata and Ananas comosus) and dicotyledoneous (Arabidopsis thaliana, Aquilegia coerulea, Cucumis sativus, Medicago truncatula, Carica papaya, Populus trichocarpa, Solanum lycopersicum and Ricinus comunis) and phylogenetic studies, we have not found monocotyledonous-non-grass and dicotyledonous sequences homologous to RA1.

When the peptide sequence of RA1 and its homologs with respect to other zinc finger proteins, are comparatively analyzed, it is observed that the zinc finger domain is linked to DNA by a short a-helix containing the amino acid sequence QGLGGH, with a glycine residue that relaxes the helix conserved 100% between RA1 and its counterparts. From a study carried out in this work, we detected that L can be replaced by M. On the other hand, the characteristic motif of other zinc finger proteins NNM/WQMH is absent. Furthermore, no B box type of nuclear localization signal has been observed at the N-terminus as has traditionally been identified for zinc finger proteins.

When the peptide sequence is comparatively analyzed, we note that the RA1 homologs of various grass species of PACMAD differ markedly from that of maize, especially in the number of EAR motifs. RA1 was originally described as a repressor protein with two EAR motifs (Vollbrecht et al., 2005). However, when comparing the sequences between the members of the PACMAD we noted that RA1 is made up of three EAR motifs. These data were also verified using motif searches in the Motif-based sequence analysis tool database (Bailey et al., 2009). Likewise, the modification or absence of the EAR motif closest to the zinc finger domain in the PACMAD grass sequences not related to maize is also highlighted. Recent studies on the affinity of interaction of TPL transcriptional co-repressors with transcriptional repressors indicate that, (1) the greater the number of Leucines (L) in the EAR motif, the greater the stability in the interaction with TPL, (2) the residues of final Ls confer more binding stability with TPL, (3) greater numbers of EAR motifs, greater stability of interaction with TPL, and (4) regions bordering EAR motifs are equally important in stabilizing the binding with TPL proteins (Ke et al., 2015). The differences observed in terms of numbers of EAR motifs present in the RA1 proteins of the PACMAD clade suggest a differential affinity for RA1 in the presence of a transcriptional co-repressor such as REL2. So far, it is unknown whether the difference in the number of RA1 EAR motifs affects protein-protein interaction with REL2.

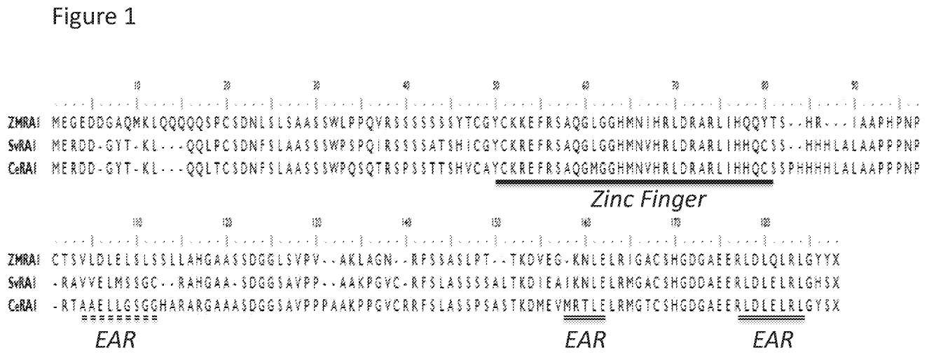

In particular, the maize, Setaria viridis and Cenchrus equinatus RAMOSA1 peptide sequence is composed of the C2H2 zinc finger domain with QGLGGH sequence and downstream are three and two EAR repressor domains (LxLxLxL) respectively (SEQ ID NO1 and SEQ ID NO2 and SEQ ID NO3) (FIG. 1). Maize RAMOSA1 has three sequences of EAR motifs VLDLELSLS, NLELRIG and RLDLQLRLG. The RAMOSA1 homolog in Setaria viridis and Cenchrus equinatus has two EAR motifs of sequence NLELRMG and RLDLELRLG.

Additionally, by means of the MEME analysis carried out in this work, three conserved auxiliary motifs of unknown function, were identified between RA1 and its counterparts (FIG. 1):

a) one motif, here called WPPP, are typically found between the N-terminal and the zinc finger. The WPPP motif is represented by the consensus sequence SWP (L) PPQhRS (1-7). Where h is a hydrophobic residue (any of A, C, F, G, H, I, K, L, M, R, T, N, W, Y)

b) a motif, here called the CSD motif, is typically found between terminal N and the WPPP motif. The CSD motif is represented by the consensus sequence Q (2-5) P (T) CSDN (T) F (L/N) L (S/F).

c) a motif, here called the PNPNP motif, is typically found between the zinc finger and the first EAR motif. The PNPNP motif is represented by the consensus sequence AAPP (H) P (S) N (P) PNP (H/N) S (G/R) R (C/S/P).

In summary: RAMOSA1 and its counterparts have the QGLGGH motif (where the first G characterizes the group and is 100% conserved between RA1 and their homologs), two or three EAR motifs at the C-terminal of the protein, a WPPP motif located between the N-terminal and the zinc finger, a CSD motif located between the N-terminal and the WPPP motif and a PNPNP motif located between the zinc finger and the first EAR.

2) Construcciones de ADN, Vectores y Transformación de E. coli y Agrobacterium.

a) Genomic DNA Extraction

The coding sequences of the RA1 genes of maize, Setaria viridis and Cenchrus equinatus were amplified from genomic DNA extracted from leaves of plants of Zea mays genotype B73, plants of Setaria viridis genotype A10.1 and collected plants of Cenchrus equinatus (Reinheimer and Bellino, Santa Fe, Argentina).

The method used to extract genomic DNA from Setaria viridis, Cenchrus equinatus and maize plants was adapted from Doyle and Doyle (1990) (Michael Mckain pers. Comm., 2016). A Setaria viridis and Cenchrus equinatus leaves or an equivalent portion of a maize leaf was used as a sample. Initially, the frozen leaves were pulverized in a mortar using liquid nitrogen. Then, 3 mL of CTAB buffer solution (CTAB 2g, 10 mL Tris pH8 1M, 4 mL EDTA pH8 5M, 1 g PVP 40, 40 mL H2O milli Q, 10 uL β mercapto per 5 mL of CTAB prepared) was added, previously heated at 65° C. The mixture was mortar until a white liquid was obtained, which was subsequently deposited in 1.5 mL tubes containing 1000 μL of the mixture each. The tubes were heated to 65° C. for one hour and mixed by inversion every 15 minutes. When the tubes reached room temperature, equal parts of phenol:chloroform:isoamyl alcohol (25:24:1) was added. Subsequently, the mixture was centrifuged at 12000 g for 15 minutes. The aqueous phase was separated and placed in a new tube to which a volume of isopropanol pre-cooled to 4° C. was added. Then 10 μL of 3M sodium acetate was added to each tube, centrifuged at 12000 g for 15 minutes and the supernatant was discarded. The precipitate was washed with cold 70% ethanol and the mixtures of each sample were combined into a single tube. The tubes were then centrifuged at 12000 g for 10 minutes, the supernatant was discarded, and the precipitates were allowed to dry for 1 hour and 15 minutes at room temperature. Finally, the samples were resuspended in 100 μL of 1K TE (10 mM Tris, EDTA pH 8 1 mM).

b) Amplification of the Complete Sequences of Maize RA1 and its Homologs in Setaria viridis and Cenchrus equinatus

The entire sequences of maize RA1 and its homolog of Setaria and Cenchrus were amplified by Polymerase Chain Reaction (PCR) using specific oligonucleotides. The oligonucleotides used in the clones were designed using the primer-BLAST server available in the NCBI database (www.ncbi.nlm.nih.gov, Ye, et. Al, 2012) (Table 1).

For the PCR, the reaction buffer provided by the manufacturer of the enzyme was used, to which the following reagents were added: 2.5 mM MgCl2, 0.25 mM dNTPs each, 0.25 μM of each specific oligonucleotide (Table 1) and the enzyme Taq DNA Polymerase (Bio-Logical Products, PB-L), at a concentration of 1.5 U of enzyme per reaction. An appropriate dilution of DNA template was incorporated into this reaction mixture. The total reaction volume was 25 μL. Amplification reactions were carried out in the IVEMA T18 thermocycler (Ivema Desarrollos SRL), and in general the following program was used, in which the hybridization temperature (Ta) was established according to the composition of the oligonucleotide bases used, applying the following relationship for its calculation:

Ta=2×(A+T)+4×(G+C)−5° C.

Program: (3 minutes at 94° C., 1 minute at Ta, 45 seconds at 72° C.) 35 cycles+10 minutes at 72° C.

TABLE 1
Oligonucleotides used for cloning.

| | Sequence | |
|---|---|---|
| Name | (5′-3′) | Use |
| RA1-Zm_EcoRI- | CCGGAATTCATG | Amplification |
| Fw | GAGGGAGAAGAT | of RA1 from |
| | GACGG | maize |
| RA1-Zm_XhoI- | GGCCTCGAGTCA | Amplification |
| Rv | GTAGTAGCCCAG | of RA1 from |
| | TCTA | maize |
| SvRA1 F | CGGGGATCCATG | Amplification |
| BAMHI | GAGAGAGATGAT | of RA1 from |
| | GGCTAC | Setaria |
| | | viridis |
| | | and |
| | | Cenchrus |
| | | equinatus |
| SvRA1 R | CGGGAATTCTCA | Amplification |
| ECORI | GGAGTGGCCAAG | of RA1 from |
| | TCTTA | Setaria viridis |
| CeRA1ECORIRv | GGCGAATTCTCA | Amplification |
| | GGAGTAGCCAAG | of RA1 from |
| | TCTAAG | Cenchrus |
| | | equinatus |
| ZmRA1-Fw- | CGGAAGCTTATG | Amplification |
| pCAMBIA- | GAGGGAGAAGAT | of RA1 from |
| HindIII | GACGG | maize |
| ZmRA1-Rv- | CCGACTAGTTCA | Amplification |
| pCAMBIA-SpeI | GTAGTAGCCCAG | of RA1 from |
| | TCTAAG | maize |

c) DNA Electrophoresis on Agarose Gels

For the analysis of DNA fragments in agarose gels, the horizontal electrophoresis system was used (Ausubel et al., 1987). Agarose concentration was 1% (w/v). Gels were prepared in TAE 1× solution (20 mM Tris-Ac (pH 8.5), 1 mM EDTA). Prior to loading the gel, each sample was supplemented with Loading Buffer solution (Bromophenol blue 0.25% w/v, xylencianol FF 0.25% w/v, glycerol 30% v/v) in a 1/10 ratio (v/v). The electrophoretic runs were carried out in TAE 1× solution, with constant voltage between 1 and 5 V/cm of gel. Visualization of the DNA fragments was performed on an ENDURO GDS UV light transilluminator (Labnet, California, USA). To estimate the length of the DNA fragments electrophoretically separated, the molecular weight marker obtained by digesting genomic DNA of bacteriophage λ with the restriction enzyme HindIII, whose product is an equimolar mixture of DNA fragments from the DNA, was seeded in the same gel. 23130, 9416, 6557, 4361, 2322, 2027, 564 and 125 bp.

d) Purification of DNA Fragments on Agarose Gels.

In order to purify the DNA fragments in agarose gels, the gel was placed on a UV transilluminator previously cleaned with alcoholic solution and the bands of interest were cut with a sterile blade. For the extraction of the PCR product, the commercial gel purification kit (TRANSgenes) was used, following the protocol recommended by the manufacturer.

e) Cloning Into Entry Vector and pCAMBIA Expression Vector

The amplified and purified fragments were cloned, by enzymatic digestion and subsequent ligation, into the pENTR3C entry vector designed for cloning with the Gateway system (Mann et al. 2012). This vector contains i) a multiple cloned site surrounding a bacterial death cassette (ccdB), ii) a kanamycin resistance cassette in bacteria, and iii) an origin of replication in bacteria.

In addition, the amplified and purified fragments of maize RA1 were cloned, by enzymatic digestion and subsequent ligation, into the pCAMBIA expressing vector. This vector contains i) 1100 bp of the maize Ubiquitin promoter, ii) a NOS terminator, iii) a kanamycin resistance cassette in bacteria, and iii) an a hygromicyn resistance in plant.

For this, DNA digestion with restriction endonucleases was carried out following the reaction conditions recommended by the supplier (Promega). In all cases, 1-5 U of enzyme were used for each microgram of DNA to digest and it was incubated 3 hours at 37° C. supplying the total volume of the enzyme twice (at time zero and half the incubation time). BAMHI and ECORI enzymes were used for cloning SvRA1 and CeRA1. XHOI and ECORI enzymes were used for the cloning of ZmRA1 into pENTR3C entry vector. HINDIII and SPEI enzymes were used for the cloning of ZmRA1 into pCAMBIA expression vector. Ligation of the DNA fragments was carried out using 1U T4 DNA ligase (Promega), in a reaction volume of 10 μL using the reaction buffer provided by the enzyme supplier. Insert and vector quantities were used such that the molar ratio between the two was 3:1. Incubation was performed at 4° C. ON (overnight). Then, competent E. coli cells were transformed by electroporation. For the transformation of the bacteria with the corresponding vector, the electric shock was carried out in 0.2 cm cuvettes (Bio-Rad). Immediately after discharge, 1 mL of LB culture medium (meat Peptone 10 g/L, yeast extract 5 g/L, NaCl 5 g/L; pH 7) was added.

The cell suspension and the mixture were incubated for 1 hour at 37° C. After centrifuging at 4500 g for 5 minutes, the cell pellet was resuspended in 50 μL of LB medium and inoculated in Petri dishes containing LB agar culture medium (LB plus 15 g/L agar) supplemented with the appropriate antibiotics. The plates were incubated at the corresponding temperature for each bacterium until the appearance of individual colonies (approximately 16 hours for E. coli DH5α). It is important to note that the entire process was carried out under sterile conditions using a horizontal air flow cabin. The material used was autoclaved for 20 minutes at 1 pressure atmosphere and 120° C.

The purification of DNA plasmid from the bacteria culture was performed using the alkaline lysis method (Bimboim et al., 1979). Transformed cells were grown ON at 37° C. with shaking (180 rpm) to saturation in LB culture medium supplemented with the corresponding antibiotic. For each preparation, 1.5 mL of the saturated culture was centrifuged at 12000 rpm for 1 minute at room temperature. The cell pellet was completely resuspended in 100 μL of solution I (25 mM Tris-HCl pH8, 10 mM EDTA) and incubated on ice for 5 minutes. Then 200 μL of solution II (0.2M NaOH, 1% w/v SDS) was added, tubes were mixed by inversion and incubated on ice for 5 minutes. Then 150 μL of solution III (5M Potassium Acetate pH 4.8) was added, tubes were mixed again by inversion and incubated on ice for 5 minutes.

Subsequently, the mixture was centrifuged at 12000 rpm for 10 minutes at 4° C., recovering the supernatant to which an extraction with chloroform/isoamyl alcohol (24:1) was performed. After vigorous vortex, it was centrifuged at 8000 rpm for 5 minutes at room temperature. The aqueous phase was again recovered and the plasmid DNA present in it was precipitated by adding 0.8 volumes of isopropanol, followed by incubation at −20° C. for 10 minutes and centrifugation at 12000 rpm and 4° C. for 15 minutes. The precipitate was washed with 800 μL of 70% (v/v) ethanol, to remove salts, and centrifuged at 12000 rpm for 5 minutes at room temperature. The supernatant was discarded and the precipitate was allowed to dry at room temperature. Finally, it was resuspended in 30 μL of sterile mili Q ultrapure water and 1 μL of RNase was added for the elimination of bacterial RNA residues. The purification results were verified by means of DNA electrophoresis on agarose gels following the described method.

After confirming the identity of the cloning sequences by sequencing, the entry vector was recombined with a destination vector.

f) Cloned Into Destination Vector

The entry vector was recombined with a destination vector designed for cloning by Gateway system, pANIC 6A (Mann et al., 2012), using LR clone II (Life Technologies). The pANIC 6A vector is a target vector designed for cloning using the Gateway system, which allows overexpression of sequences of interest in monocotyledons (Mann et. al, 2012). This vector contains i) a cassette compatible with the Gateway system for overexpression of the gene of interest using the maize Ubiquitin promoter (ZmUbi1), ii) a plant selection cassette (hph: Hygromycin B resistance) to confer resistance to transformed plants, and iii) a cassette containing a reporter gene (pporRFP: Porites porites red fluorescent protein) for the visual identification of transgenic plants (Mann et. al, 2012). Other relevant sequences within the vector are: the bacteria kanamycin resistance genes (Kanr), the bacteria death cassette (ccdB), the origin of replication in Escherichia coli (ColE1) and in Agrobacterium tumefaciens (PVS1). This vector is a plant integration vector, since after transformation, the vectors integrate a part of the vector DNA into the genome of the host plant.

The in vitro recombination reaction of DNA fragments was carried out using 1 μL of LR clonase II (Life Technologies), 0.75 μL of the target vector (150 ng/μL), 1.25 μL of TE solution pH6, and 2 μL of the input vector (150 ng/μL). Incubation was performed at room temperature ON. The 5 μL were used to transform competent E. coli DH5a cells.

For the transformation of the bacteria with the cloning into the destination vector, the electric shock was carried out following the methodology described above. Purification of plasmid DNA from the bacteria culture and its visualization was carried out using the method described above. After confirming the identity of the clone sequences in the destination vector by sequencing, competent Agrobacterium tumefaciens EHA105 cells were transformed by means of electric shock. For the transformation of the bacteria with the cloning into the destination vector, the electric shock was carried out in 0.2 cm cuvettes (Bio-Rad). Immediately after discharge, 1 mL of LB culture medium was added to the cell suspension and the mixture was incubated for 2 hours at 37° C. After centrifugation at 4500 g for 5 minutes, the cell pellet was resuspended in 50 μL of LB medium and inoculated in Petri dishes containing agarose LB culture medium supplemented with the appropriate antibiotics. The plates were incubated at the corresponding temperature for each bacterium until the appearance of individual colonies (approximately 48 hours). It is important to note that the entire process was carried out under sterile conditions using a horizontal air flow cabin. The material used was autoclaved for 20 minutes at 1 pressure atmosphere and 120° C.

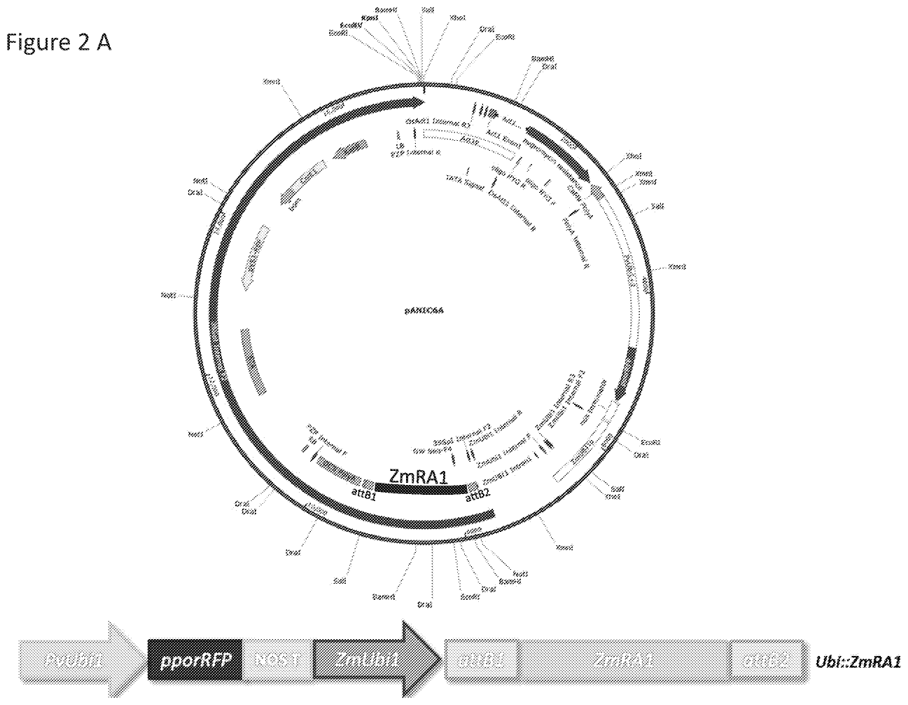

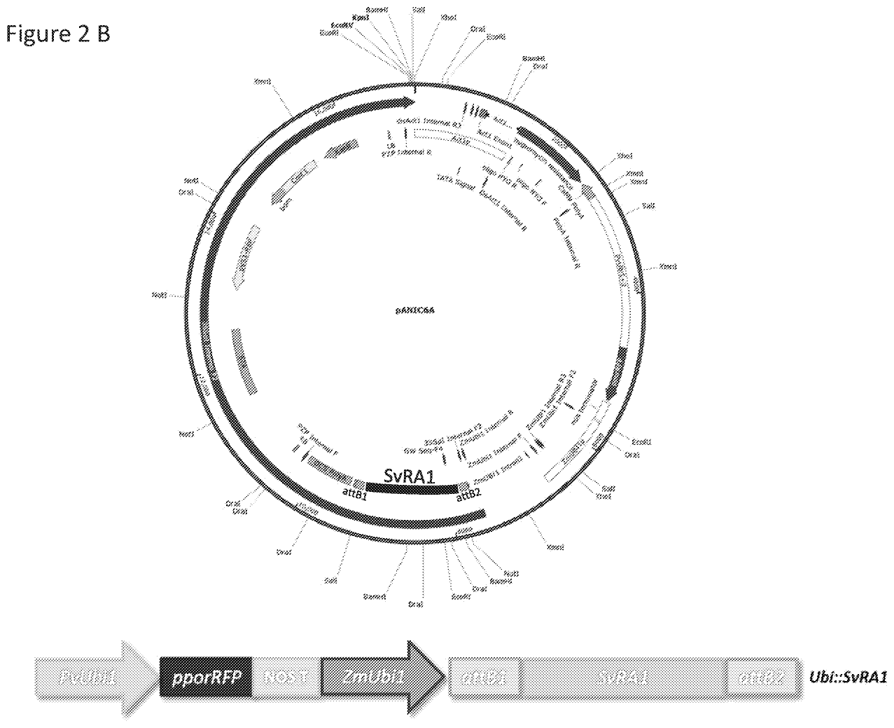

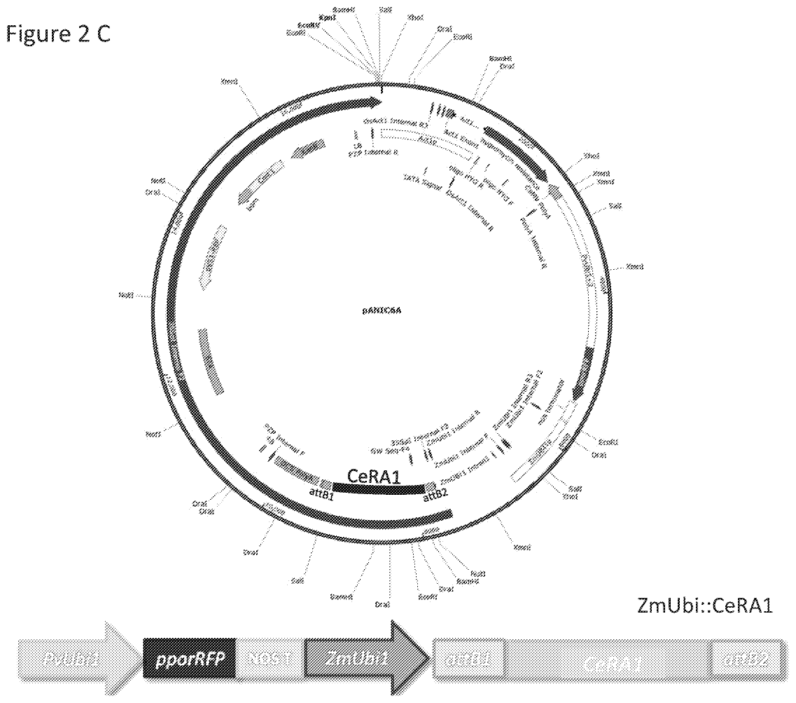

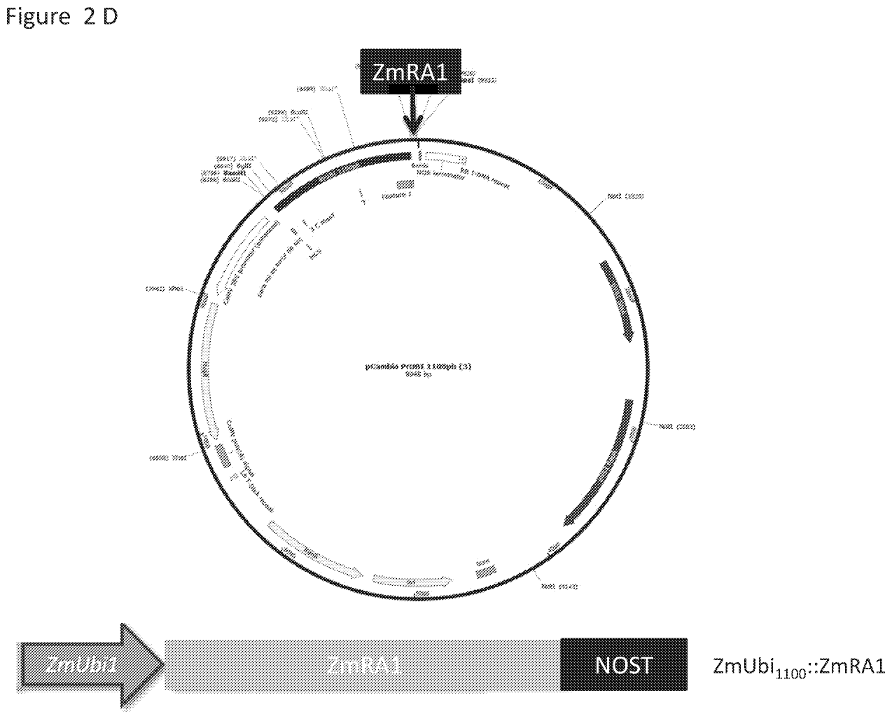

The genetic constructs used are described below: ZmUbi::ZmRA1: To generate this construct, the coding region corresponding to ZmRA1 was amplified from DNA (taking advantage of the lack of introns in the sequence) with the specific oligonucleotides RA1-Zm_EcoRI-Fw and RA1-Zm_XhoI-Rv (detailed sequence in Table 1). Then, the amplified fragment was cloned, by means of cuts with the restriction enzymes EcoRI and Xhol followed by ligation, into the entry vector pENTR 3C. Both the amplified region and the vector have a single restriction site for the mentioned enzymes, thus ensuring the correct orientation of ligation of the insert in the vector. Subsequently, the vector was recombined with the destination vector pANIC 6A by the Gateway system using the enzyme LR clonase II (Life Technologies). The result of the recombination is illustrated in FIG. 2A.

ZmUbi::SvRA1: To generate this construct, the coding region corresponding to SvRA1 was amplified from DNA (taking advantage of the lack of introns in the sequence) with the specific oligonucleotides SvRA1 F BAMHI and SvRA1 R ECORI (sequence detailed in Table 1). Then, the amplified fragment was cloned, by means of cuts with the restriction enzymes BamHI and EcoRI followed by ligation, into the entry vector pENTR 3C. Both the amplified region and the vector have a single restriction site for the mentioned enzymes, thus ensuring the correct orientation of ligation of the insert in the vector. Subsequently, the vector was recombined with the destination vector pANIC 6A by the Gateway system using the enzyme LR clonase II (Life Technologies). The result of the recombination is illustrated in FIG. 2B.

ZmUbi::CeRA1: To generate this construct, the coding region corresponding to CeRA1 was amplified from DNA (taking advantage of the lack of introns in the sequence) with the specific oligonucleotides SvRA1 F BAMHI and CeRA1ECORIRv (sequence detailed in Table 1). Then, the amplified fragment was cloned, by means of cuts with the restriction enzymes BamHI and EcoRI followed by ligation, into the entry vector pENTR 3C. Both the amplified region and the vector have a single restriction site for the mentioned enzymes, thus ensuring the correct orientation of ligation of the insert in the vector. Subsequently, the vector was recombined with the destination vector pANIC 6A by the Gateway system using the enzyme LR clonase II (Life Technologies). The result of the recombination is illustrated in FIG. 2C.

ZmUbi1100pCAMBIA ZmRA1: To generate this construct, the coding region corresponding to ZmRA1 was amplified from DNA (taking advantage of the lack of introns in the sequence) with the specific oligonucleotides ZmRA1-Fw-pCAMBIA-HindIII and ZmRA1-Rv-pCAMBIA-SpeI (sequence detailed in Table 1). Then, the amplified fragment was cloned, by means of cuts with the restriction enzymes HindIII and SpeI followed by ligation, in the expression vector pCAMBIA. Both the amplified region and the vector have a single restriction site for the mentioned enzymes, thus ensuring the correct orientation of ligation of the insert in the vector. The result of the recombination is illustrated in FIG. 2D.

3) Stable Transformation of Arabidopsis and Phenotype of Transgenic Plants

a) Stable Method of Transformation of Arabidopsis.

The method used to transform Arabidopsis plants was floral immersion with Agrobacterium tumefanciens (Clough and Bent, 1998). For this, 16 pots (8 cm in diameter by 7 cm high) were grown, in long day conditions, in a growth chamber (16 hours of light, 8 hours of darkness, at 24-22° C., humidity 50-70% and intensity of light ˜150 micromoles/m2/sec.) with three to four Arabidopsis plants each, until flowering (approximately four weeks). When the flower stalks grew large enough to separate from their proximal axillary bud, the inflorescences were cut without damaging the caulinal leaves and nearby axillary buds. Between two and three days after cutting, new inflorescences emerged from the axillary buds, which were cut again taking the aforementioned care. The transformation was carried out three days after the last cut.

To prepare the transformation suspension, A. tumefanciens cells, containing the specific vector for overexpression, were cultured for 16 hours at 28° C. with shaking, in flasks containing 10 mL of LB culture medium supplemented with rifampicin antibiotic (2 pL / mL) and kanamycin antibiotic (1 μL/mL). These cultures were used to inoculate 200 mL of the same medium supplemented with the same antibiotics contained in an Erlenmeyer flask. The cells were cultivated until reaching the stationary phase under the same conditions as the previous culture process. They were then centrifuged at 4500 g for 15 minutes at 4° C. The pellets were resuspended in 500 mL of a sucrose solution (50 g/L) containing 500 μL of Silwet detergent (PhytoTechnologies Laboratories). The plants were immersed in the solution for 1 minute, trying to prevent the immersion solution from contacting the soil and the leaves of the rosette. The pots were then placed horizontally on a tray, covered with plastic wrap, and placed in the culture chamber. The next day, the pots were placed upright and watered and fertilized with Akhaphos 50 g/L (3 mL/L) solution. The plants were cultivated until the time of harvest (approximately 6-8 weeks after planting). The harvested seeds were kept at 4° C. until use.

b) Stable Method of Transformation of Rice.

The genetic contruction ZmUbi1100pCAMBIA::ZmRA1 was introduced into Oryza sativa L. ssp. Japonica cv. Kitaake (the cv. Nipponbare can be used as well) using the Agrobacterium-mediated co-cultivation method. Seeds sterilization, callus induction, co-cultivation with Agrobacterium EHA105, transformed calli selection and regeneration were carried out following established protocols (Main et al. 2015). Seven hundred calli were induced and transformed with Agrobacterium EHA105 carrying ZmUbi1100pCAMBIA ZmRA1 construct. Resistant calli were selected using Carbenicillin (100 mg/L) and Timentin (150 mg/L) antibiotics in selection media (Main et al., 2015). Resistant calli were then transfer to regeneration media I with Carbenicillin (100 mg/L) and Timentin (150 mg/L) antibiotics. (Main et al., 2015). Regenerated plantlets were transfer to a regeneration media II without the selective agents (Main et al., 2015). Calli at induction, selection and regeneration were cultivated in a growth chamber (16 hours of light, 8 hours of darkness, at 28° C., humidity 50-70% and intensity of light 150 micromoles/m2/sec.). Regenerated plants (T0) were obtained and transferred, one per pot, at the greenhouse for rustication (with 30° C./25° C. (day/night) and 16 h light/8 h dark at 50%-60% humidity with a light intensity of 20,000-25,000 lux). Plants were irrigated and fertilized with Basafer Plus (0.5 grs/L) once a week and supplemented with Basacote Plus 6M (5 grs/L) once.

c) Selection of Arabidopsis and Rice Transformants.

For the selection of Arabidopsis and rice transformants, the seeds of the transformed Arabidopsis and rice plants with the plasmid were sterilized and inoculated in Petri dishes containing MS 1K medium (Murashige and Skoog Basal Medium (Phytotechnology Laboratories) MS 2.2 g/L salt mixture (1K) Agar 0.8%) supplemented with the antibiotic hygromycin B (2 μL/mL). The plates were cultivated in a plate culture chamber for two weeks where the cotyledons of the antibiotic-sensitive plants began to turn yellow and their growth stopped, causing the death of the seedling, while the transgenic seedlings continued their normal development. Only resistant plants, which presented a root of adequate size, were transplanted into pots for the different tests to be carried out. Subsequently, the transgenic plants were individually harvested for subsequent analysis. Genomic DNA extraction was carried out on these plants to verify the presence of the transgene.

The method used to extract genomic DNA from rice plants was adapted from Doyle and Doyle (1990) (Michael Mckain pers. Comm., 2016) as explained above. Extraction of genomic DNA from Arabidopsis plants was performed by adding 100 mg of cold, pulverized leaf tissue to a tube containing 600 μL of CTAB buffer for Arabidopsis (CTAB 2%, 1.4M NaCl, 100 mM Tris pH8, EDTA 20 mM, β mercapto 0.2%, H2O milli Q) preheated to 65° C. and incubated at 65° C. for 30 minutes, shaking every 10 minutes. Then 1 volume of chloroform: isoamyl alcohol (24:1) was added and stirred for 15 seconds. The tubes were centrifuged at 500 g for 10 minutes. The aqueous phase was recovered and 0.6 volumes of isopropanol were added. The tubes were mixed by immersion and the DNA was allowed to precipitate at −20° C. for 20 minutes. The tubes were then centrifuged at 12000 g for 15 minutes, the supernatant was discarded and the precipitate was washed with 1 mL of 70% ethanol by centrifuging the tubes at 12000 g for 5 minutes. Finally, the supernatant was discarded, the precipitate was allowed to dry and it was resuspended in 50 μL of sterile mili Q ultrapure water, heating the tubes to 70° C. for 10 minutes.

A PCR reaction was made with the product of this extraction, using the oligonucleotides HYG-F (5′-CAATGACCGCTGTTATGCGG-3′) and HYG-R (5′-CTCGGAGGGCGAAGAAGAATCTC-3′) and the corresponding program ((3 minutes at 94° C., 1 minute at Ta, 30 seconds at 72° C.) 35 cycles+5 minutes at 72° C.) to identify the transformed plants. With the lines that gave positive results, we proceeded to obtain plants from the following generations to carry out their phenotyping.

d) Quantification of Transgene Expression Levels in Transgenic Plants.

Total RNA extractions were performed with the TriPure solution reagent (Roche) following the manufacturing instructions. Total RNA was extracted from the leaf of transformed Arabidopsis plants.

The quality and quantity of RNA or DNA were evaluated with the Nanodrop 2000 kit (ThermoScientific) by measuring the absorbance at 260 nm (Sambrook et al., 1989), in which the A260 value of 1 corresponds approximately to 40 μg/mL of RNA or 50 μg/mL DNA. A volume of 1 μL per sample was used for each measurement. The proteins contamination of the purifications was evaluated by means of the A260/A280 ratio, and that of carbohydrates and phenolic compounds by the A260/A230 ratio.

With the extracted RNA, reverse transcription reactions were carried out. Reverse transcription reactions were carried out in two steps: 1 μg of RNA was placed in a PCR tube and a final volume of 4.6 μL was made up with sterile mili Q ultrapure water. The tubes were incubated for 5 minutes at 65° C., and then immediately placed on ice (this treatment allows the RNA secondary structures to be disassembled). Next, 1.5 μL of enzyme buffer, 0.75 μL of dNTPs (10 mM), 0.35 μL of oligo dT (100 mM) and 0.3 μL of reverse transcriptase M-MLV 200 U/μL (Thermo Scientific) were added to each tube. This mixture was incubated 60 minutes at 42° C. and then the enzyme was inactivated by heating the reaction for 10 minutes at 70° C.

The quantification of the transcripts was carried out by means of real-time PCR. Quantitative real-time PCR (q-PCR) was carried out using a SteponePlus48 thermocycler (Applied Biosystems). Reactions were performed in final volumes of 10 μL containing 0.5 μL of forward oligonucleotide, 0.5 μL of reverse oligonucleotide, 3 μL of sterile mili Q ultrapure water and 5 μL of Syber green dye Master Mix (BioRad). The emitted fluorescence was continuously recorded for 40 cycles. The sequences of the oligonucleotides used are detailed in Table 2. For Arabidopsis, the expression levels of the PP2A gene were jointly quantified to normalize the expression levels of the genes of interest. For rice, the expression levels of the UBI gene were jointly quantified to normalize the expression levels of the genes of interest. All the quantifications were carried out with biological triplicates and technical triplicates.

TABLE 2
Oligonucleotides used in RT-qPCR reactions.

| | Sequence | |
|---|---|---|
| Name | (5′-3′) | Use |
| ZmRA1-Fw-qPCR | GTATTGCTGC | Quantification |
| | TCCCCATCCA | of ZmRA1 |
| | | expression in |
| | | Ubi::ZmRA1 |
| | | plants. |
| ZmRA1-Rv-qPCR | ACTGGAACAG | Quantification |
| | ACAAGCCTCC | of ZmRA1 |
| | | expression in |
| | | Ubi::ZmRA1 |
| | | plants. |
| SvRA1-Fw-qPCR | ACTCCTGAGA | Quantification |
| | CGACGGACA | of SvRA1 and |
| | | CeRA1 expression |
| | | in Ubi::SvRA1 |
| | | and Ubi::CeRA1 |
| | | plants. |
| SvRA1-Rv-qPCR | GGCAGAAGAA | Quantification |
| | GGCACACAAT | of SvRA1 and |
| | A | CeRA1 expression |
| | | in Ubi::SvRA1 |
| | | and Ubi::CeRA1 |
| | | plants. |
| PP2A-FW | CCTGCGGTAA | Housekeepping |
| | TAACTGCATC | used in |
| | T | RT-qPCR |
| PP2A-RV | CTTCACTTAG | Housekeepping |
| | CTCCACCAAG | used in |
| | CA | RT-qPCR |
| UBQ-Fw Grass | GCAAGAAGAA | Housekeepping |
| | GACCTACACC | used in |
| | AAG | RT-qPCR |
| | | for rice |
| | | transgenic |
| | | plants |
| UBQ-Rv Grass | CCTTCTGGTT | Housekeepping |
| | GTAGACGTAG | used in |
| | GTG | RT-qPCR |
| | | for rice |
| | | transgenic |
| | | plants |

e) Phenotypic Characterization.

Arabidopsis transgenic seeds, after being incubated at 4° C. for at least two days to break dormancy, were germinated directly in soil using 8 cm diameter by 7 cm high pots. Four seeds per pot or one seed per pot were sown according to the experiment to be performed. In all cases, the tray was covered with plastic wrap to generate the conditions of the humid chamber and facilitate germination. The plants were grown in a long-day photoperiod growth chamber (16 hours of light and 8 hours of darkness), at 24-22° C., humidity 50-70% and intensity of light ˜150 micromoles/m2/sec. After five days in a humid chamber, the plastic wrap was removed and irrigation and fertilization with Akhaphos solution (3 mL/L) began once a week.

Rice transgenic homozygote seeds were germinated directly in soil using 15 cm diameter by 18 cm high pots. Four seeds per pot or one seed per pot were sown according to the experiment to be performed. Seedlings were grown in a greenhouse with 30° C./25° C. (day/night) and 16 h light/8 h dark at 50%-60% humidity with a light intensity of 20,000-25,000 lux. Plants were irrigated three times a week and supplemented with Basacote Plus 6M (5 grs/L) once.

ANOVA was used for the statistical treatment of the data, using the LSD test (Least Significant Differences) with a significance level of 5%.

Results:

Characterization of the Phenotypes of Transgenic Arabidopsis Ubi SvRA1 and CeRA1 Plants:

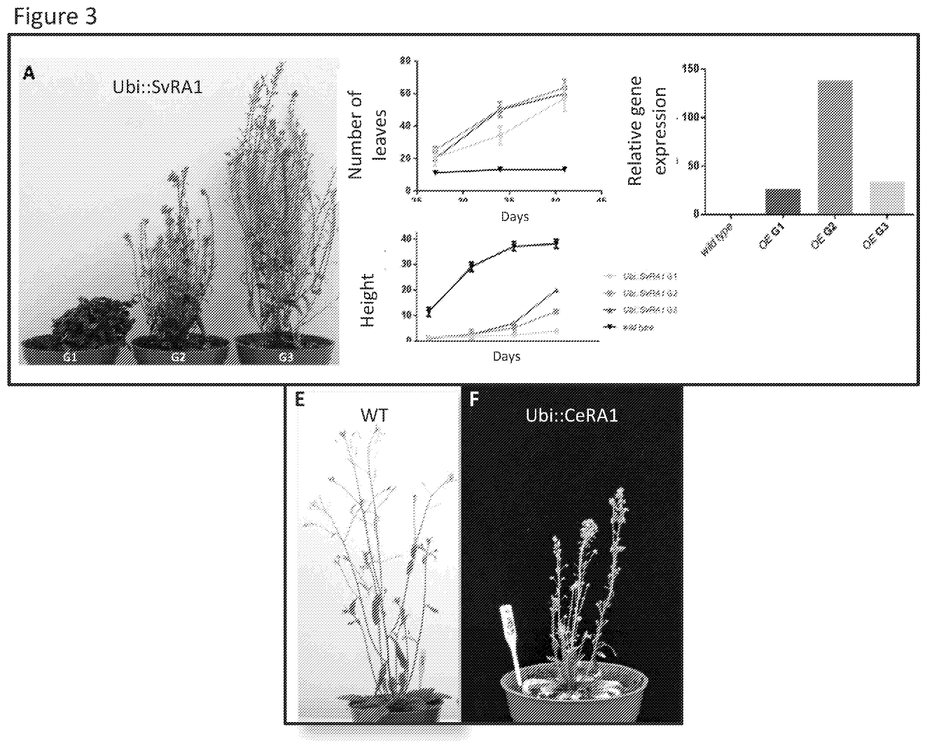

The Arabidopsis transgenic lines used in the different experiments correspond to homozygous plants with relatively low levels (G1), intermediates (G3) and high levels of expression (G2) (FIG. 3A-D). Transgenic plants with intermediate levels of expression were identified as L10-G3. Transgenic plants with high levels of expression were identified with L4-G2, L5-G2, L6-G2, L7-G2, L8-G2, L9-G2.

The Arabidopsis transgenic plants overexpressing SvRA1 and CeRA1 have similar phenotypes (FIG. 3A-F). SvRA1 and CeRA1 overexpression generates plants with an increased life cycle, biomass, greater number of leaves, greater coverage area above the ground, lower height, higher growth rate of roots, lower sensitivity of roots to biotic stress.

I) Ubi::SvRA1 and Ubi::CeRA1 plants show, from the beginning of germination, an increase in the number of leaves and a decrease in height with respect to the wild control plants, regardless of the level of expression of the transgene.

The number of leaves was counted every ten days after the appearance of the cotyledons above the ground until reaching 45 days. The data obtained from these measurements are summarized in FIG. 3A-B.

The height of the plant was calculated using photographs taken every ten days up to 50 days after germination. The height of the plant was determined by means of the distance between the horizontal lines that go through the upper edge of the pot and the highest pixel that corresponds to a part of the plant above the ground. This value was converted by calibration, as a physical distance expressed in centimeters. The results of the maximum values of the height above the ground of the lines selected for the evaluation are summarized in FIG. 3A, C. According to the results of FIG. 3, it can be concluded that:

i—Transgenic plants have 3.5 times more leaves than wild plants.

ii—Transgenic plants are 5 times less taller than wild plants.

II) Ubi::SvRA1 plants show an increase in aboveground biomass.

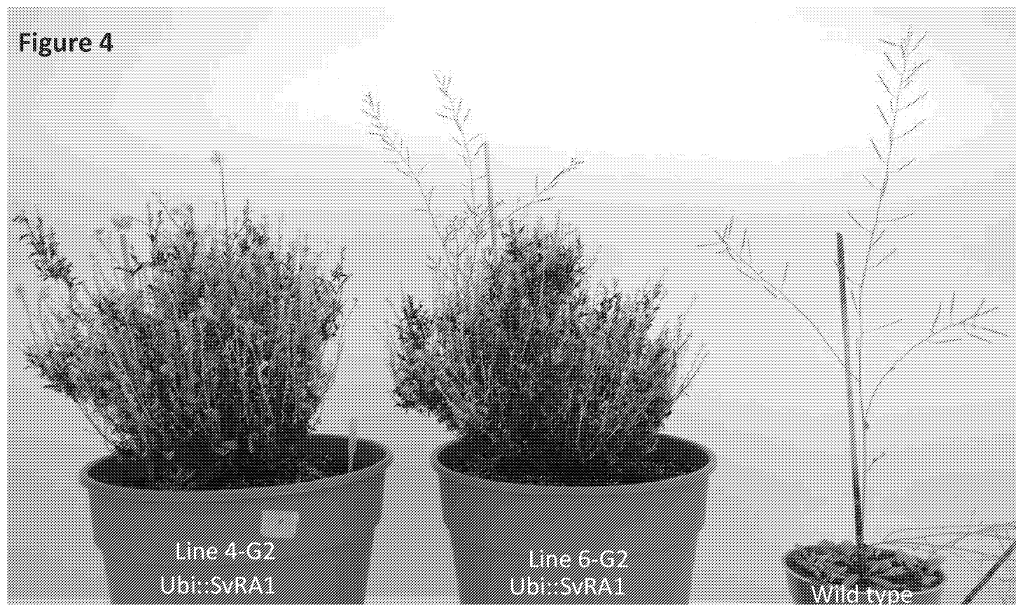

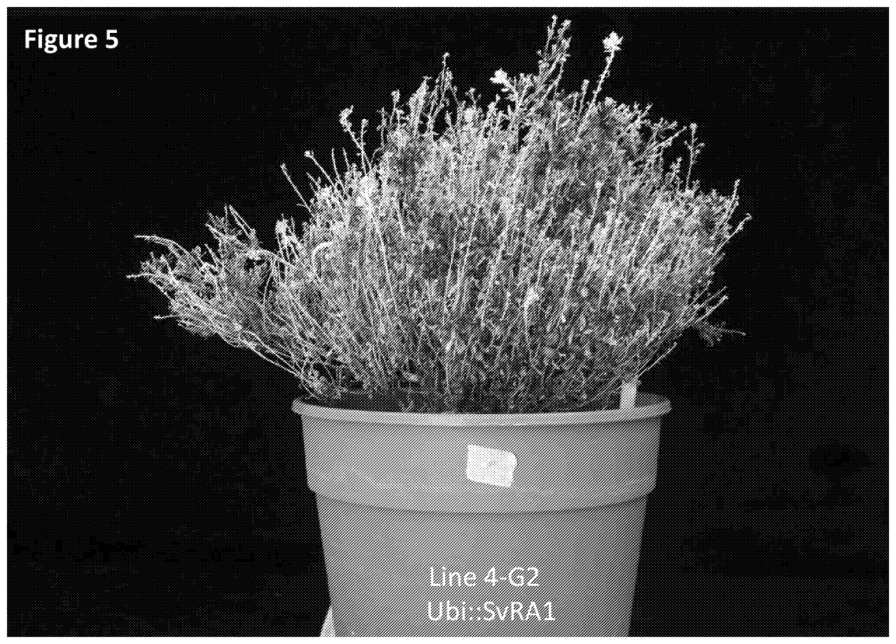

FIG. 4 documents the increase in aboveground biomass. FIG. 4 shows two lines of transgenic plants at the middle of the life cycle (6 months) compared to wild control plants at the end of the life cycle (2 months). FIG. 5 documents line 4-G2 at the end of the life cycle.

To quantify the differences in biomass at the end of the life cycle, dry weight measurements were obtained from the aerial part of the transgenic lines and wild plants. The tissues were harvested, dried and weighed using a precision balance. According to Table 3, it can be concluded that:

The plants of the present invention are characterized by having an increase of 29 to 44 times or more in aerial biomass with respect to wild plants at the end of the life cycle.

TABLE 3
Dry weight expressed in grams (grs) at the end of the life
cycle in three transgenic lines and wild control plants. The differences
in dry weight of aerial biomass between the transgenic lines and the
wild plants expressed in grams (dif), number of times, and percentage
are presented.

| | Dry weight of | | | |
|---|---|---|---|---|
| | aerial part at | | | |
| | the end of the | | | |
| | life cycle | | Increment (n° | |
| | (grs) | dif | of times) | Increment (%) |
| wt | 0.25 | | | |
| L4-G2 | 11 | 106635 | 44 | 43654 |
| L5-G2 | 7 | 69802 | 29 | 289208 |
| L6-G2 | 102 | 975 | 40 | 4000 |

III) Ubi::SvRA1 and Ubi::CeRA1 Plants Show an Increase in the Life Cycle Extension.

In order to quantify the extension in the life cycle, the number of days of life from the first day of emergence of the cotyledons over the substrate has been quantified. Table 4 documents the increase in the number of days of life of the plants of the present invention with respect to wild plants. Table 5 exemplifies the relationship between life cycle and biomass production. According to Tables 4 and 5, it can be concluded that:

i—Transgenic plants show a significant increase of 6.5 or more lives compared to wild plants.

ii—For each life, transgenic plants show a significant increase in average above-ground biomass production between 4.4 and 6.7 or more times compared to wild plants.

iii—The production of aerial biomass per day is significantly higher in transgenic plants compared to wild control plants.

TABLE 4
Life cycle duration in different transgenic lines and wild
control plants. Differences in life cycle duration expressed in
months, days and number of additional lives are presented in relation
to the duration of life of wild control plants.

| | Increment of the life cycle extension | | |
|---|---|---|---|
| | (months) | days | lifes |
| Wt | 2 | 60 | |
| L4-G2 | 13 | 392 | 6.5 |
| L5-G2 | 13 | 392 | 6.5 |
| L6-G2 | 13 | 392 | 6.5 |
| L7-G2 | 13 | 392 | N/D |
| L8-G2 | 10 | 300 | 5 |
| L9-G2 | 12 | 360 | 6 |
| L10-G3 | 5-continue | 150-∞ | N/D |
| L10-G3 | 7-continue | 210-∞ | N/D |
| L11-G2 | 16-continue | 490-∞ | N/D |
| L12-G2 | 12 | 360 | 6 |
| L13-G2 | 11 | 330 | 5.5 |
| CeRA1-L1, | 4-continue | 120-∞ | N/D |
| L2, L4-10 | | | |

Abbreviations: ∞, continues;
N/D, not determined.

TABLE 5
Life cycle duration in three transgenic lines and wild control
plants and their relationship with the increase in biomass.
Differences in life cycle duration expressed in
months, days and number of additional
lives are presented in relation to the life extension of wild
control plants. The relationship between life
cycle duration and aboveground biomass
production, expressed in grams (grs), was calculated from
the data presented in Table 3. The results between the increase
in lifecycle duration and the biomass production was evaluated
based on the increase in grams per additional life and per day.

| | Increment in | | | Gained | Increment of | |
|---|---|---|---|---|---|---|
| | the extension | | | grs per | gained grs | |
| | of the life | | | life | per life | Gained |
| | cycle | | | (aver- | (number of | grs per |
| | (months) | days | lifes | age) | time) | day |
| Wt | 2 | 60 | | 0.25 | | 0.0041 |
| L4-G2 | 13 | 392 | 6.5 | 1.679 | 6.7 | 0.0279 |
| L5-G2 | 13 | 392 | 6.5 | 1.11 | 4.4 | 0.0185 |
| L6-G2 | 13 | 392 | 6.5 | 1.53 | 6.12 | 0.0255 |

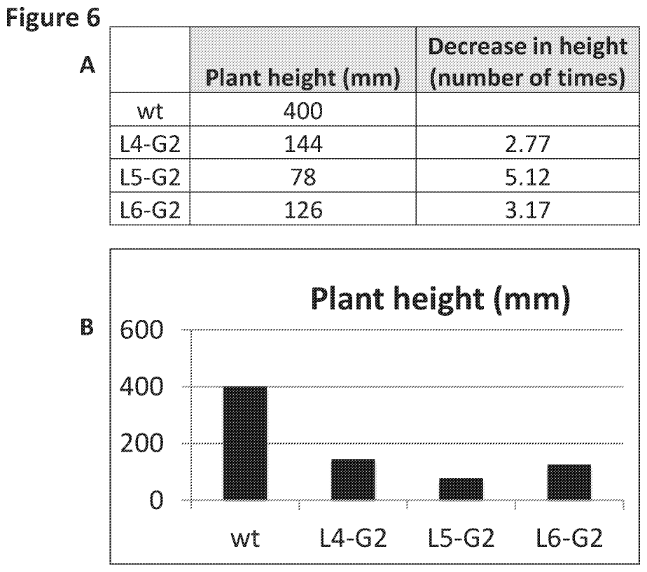

IV) Transgenic plants have lower height than wild plants. The height of the plant was calculated using photographs taken at the end of the life cycle (eg FIG. 5). The height of the plant was determined by means of the distance between the horizontal lines that go through the upper edge of the pot and the highest pixel that corresponds to a part of the plant above the ground. This value was converted by calibration, at a physical distance expressed in millimeters. The results of the maximum values of the height above the ground of the lines selected for the evaluation are summarized in FIG. 6. According to the results of FIGS. 5 and 6, it can be concluded that:

i—Transgenic plants are between 2.77 to 5.12 times shorter than wild plants.

V) The Transgenic Plants Present a Greater Covered Area Above the Ground Compared to Wild Plants.

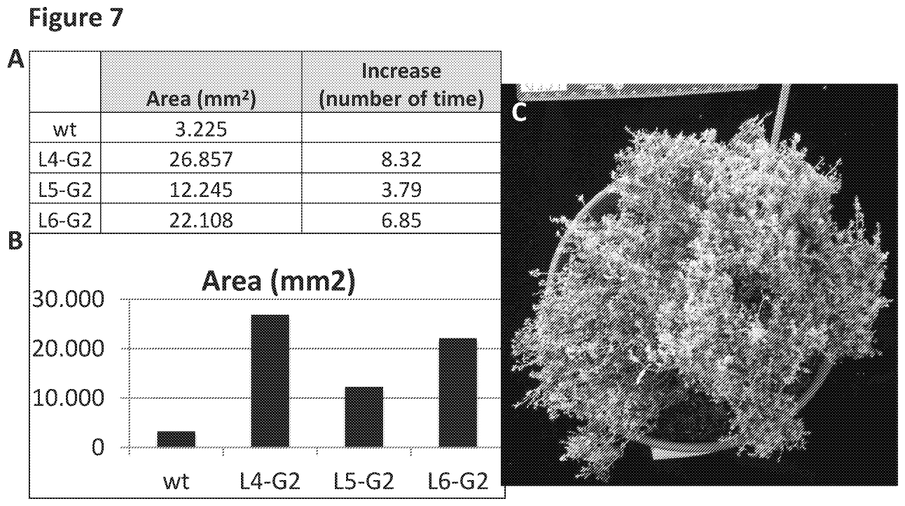

The total area of the plant above the ground was calculated using photographs taken at the end of the life cycle (eg FIG. 7). The area above the ground of the plant was determined by counting the total number of pixels from photographs of the parts of the plant above the ground discriminated from the background. This value was converted into a physical surface value expressed in square millimeters by means of calibration. The results of the maximum values of the area above the ground of the lines selected for the evaluation are summarized in FIG. 7. According to FIG. 7:

i—Transgenic plants show an increase in the area above the ground of between 3.7 to 8.3 times more compared to wild plants.

VI) The Transgenic Plants Show an Increase in Root Growth Parameters.

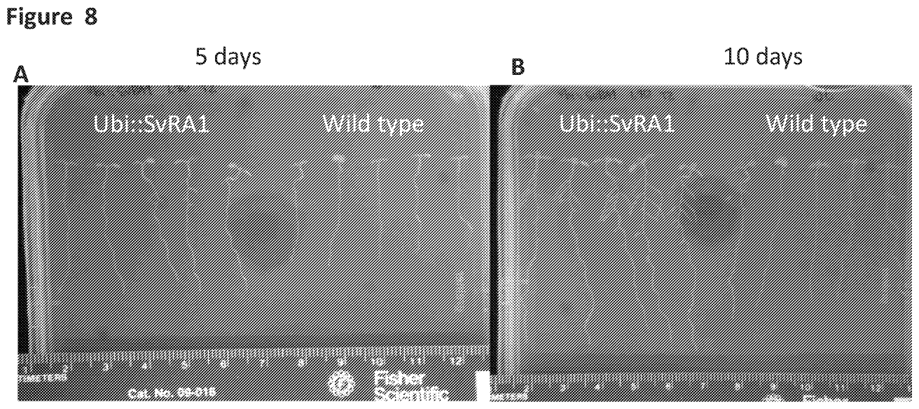

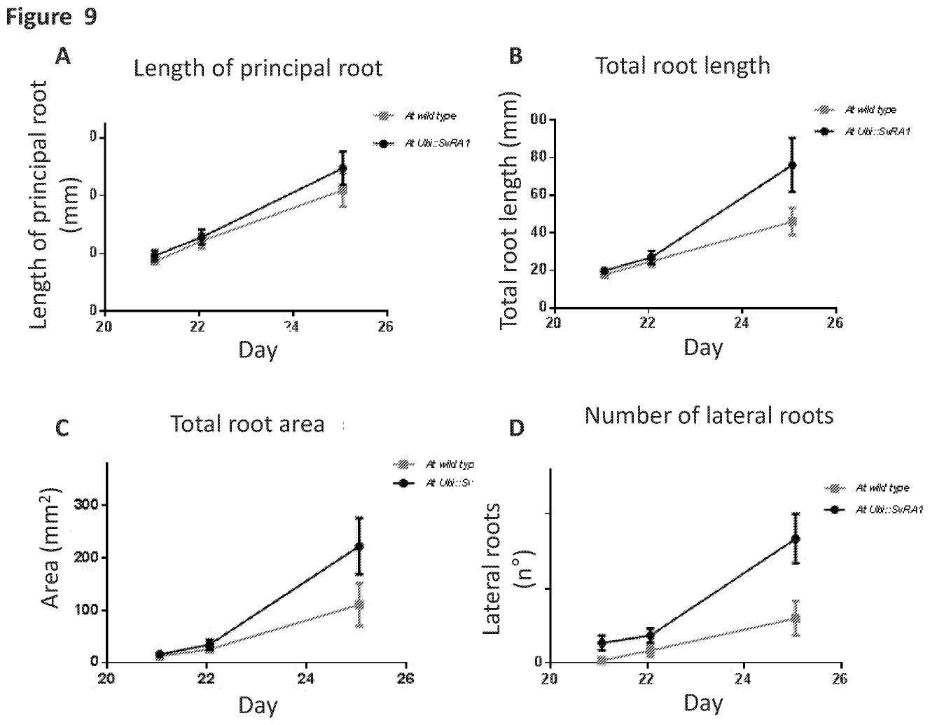

Seeds of the transgenic plants were sown alongside wild plants in MS plates. The plates were placed in an upright position in a long-day photoperiod growth chamber (16 hours of light and 8 hours of darkness), at 24-22° C. and light intensity ˜150 micromoles/m2/sec. Measurements were taken 14 days after germination. The photographs of the root were taken weekly during the growth of the plant (FIG. 8). The photographs are processed and analyzed to extract the values for the root parameters as detailed below (FIG. 9).

a—Root Area

The total area of the root is calculated from the sum of the pixels of each of the images in the root. A positive linear correlation between root area and dry weight and average root biomass has previously been established through similar experiences. Therefore, the root area is a good approximation for the root biomass.

b—Root Length

The total root perimeter of a plant is calculated as the sum of the perimeter of all the roots in the images. A linear correlation between this measurement and root length was previously established. Therefore, the root length is extrapolated from the total root perimeter.

i—The transgenic plants of the present invention show an improved development compared to the control plants. FIGS. 8 and 9 show the results of these experiments. Transgenic plants are altered by one or more root parameters as detailed above. In particular, transgenics have higher root biomass, for example, due to an increase in root area, and/or an increase in total root length.

VII) Transgenic Plants Show a Decrease in Sensitivity to Biotic Stresses.

Seeds of the transgenic plants were sown together with wild plants in MS plates inoculated with Fusarium sp. (plant pathogenic filamentous fungus).

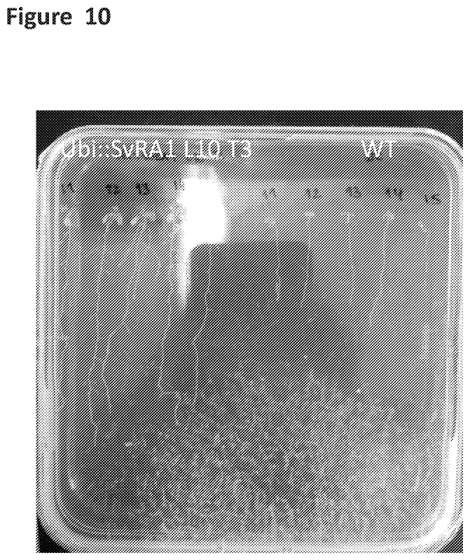

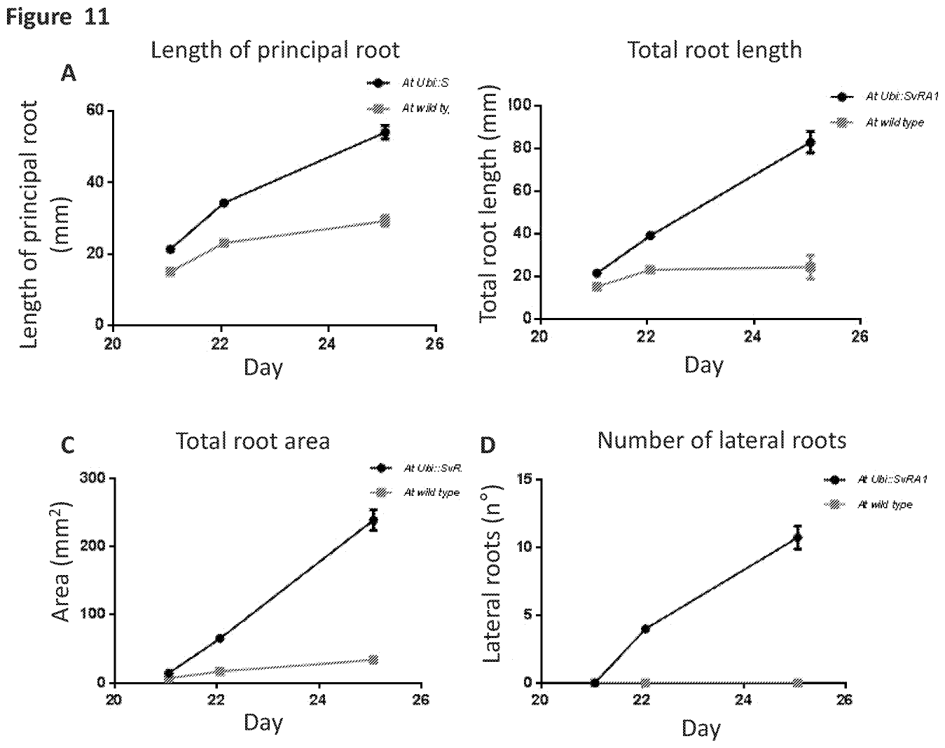

The plates were placed in an upright position in a long-day photoperiod growth chamber (16 hours of light and 8 hours of darkness), at 24-22° C. and light intensity ˜150 micromoles/m2/sec. Measurements were taken 14 days after germination. Root photographs were taken weekly during root growth up to 14 days post-germination (eg FIG. 10). The photographs are processed and analyzed to extract the values for the root parameters as detailed above. FIG. 11 shows the results of these experiments. According to the results of FIGS. 10 and 11, it can be concluded that:

i—Transgenic plants have higher root biomass, for example, due to an increase in root area, and/or an increase in root length in the presence of the pathogen compared to wild control plants.

In summary, based on the evaluation of Ubi::SvRA1 and Ubi::CeRA1 transgenic plants, it can be concluded that the presence of the SvRA1 and CeRA1 transgenes have a positive effect on the size of the plant, as well as a highly significant positive effect on the final yield of harvested biomass. These characteristics are suitable for the production of food, forage and biofuels.

Transgenic plants show characteristic traits of perennials. The SvRA1 and CeRA1 zinc finger gene can be useful for turning annuals into perennials.

The plants of the present invention have excellent characteristics of prolonged growth over time and with high production of biomass, characteristics suitable for the production of enzymes, pharmaceuticals or agrochemicals.

The plants of the present invention show an increase in root biomass, a characteristic that is particularly important in legumes (eg soybeans and alfalfa). In legumes, an increase in underground biomass promotes improvements in nitrogen fixation and nutrition from the substrate.

An improvement in the development of the root system is a desirable characteristic for any species of cereal since it promotes irrigation and aeration of the soil and prevents erosion.

The higher root biomass attenuates the effects of water stress and prevents plant dump events that considerably reduce production.

Transgenic plants are less sensitive to the attack by pathogens (e.g. Fusarium). The attack of pathogenic fungi affects most crops, causing losses and decreases in crop yields. The SvRA1 and CeRA1 zinc finger genes may be useful in decreasing sensitivity to pathogen attack in crops of interest.

In addition, a nucleic acid encoding the zinc finger protein SvRA1 and CeRA1 can be used for plant breeding programs with a view to develop higher yielding plants.

The results presented indicate that SvRA1 technology can be used to obtain plants with improved agronomic characteristics.

The overexpression of ZmRA1 generates plants that show the same height as wild plants, an increase in the number of leaves per plant, an increase in the production of seeds per silique and consequently per plant.

Characterization of the Phenotypes of Transgenic Arabidopsis Ubi ZmRA1 Plants:

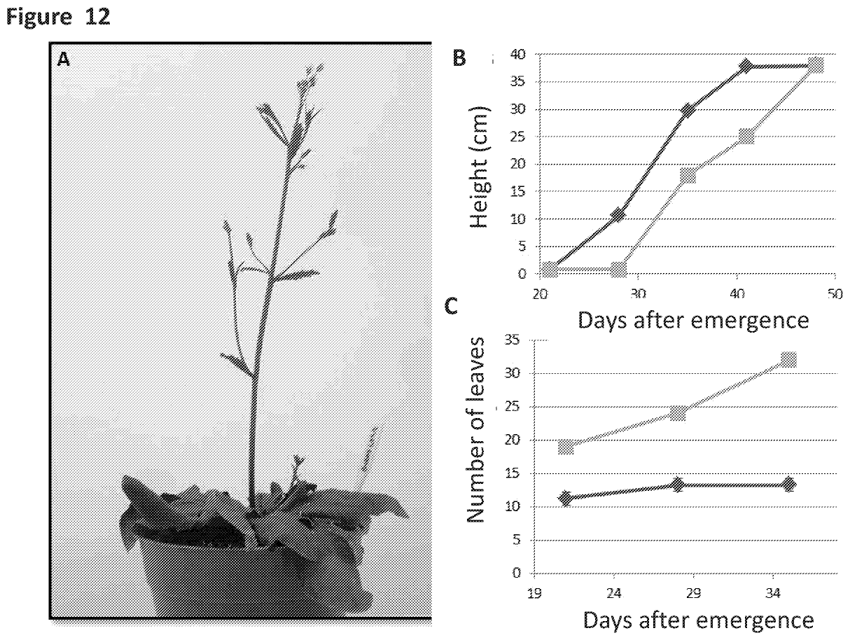

I) Ubi::ZmRA1 plants are similar in height to wild plants. FIG. 12A documents the phenotype of transgenic plants.

The height of the plant was calculated using photographs taken every ten days up to 50 days after germination. The height of the plant was determined following the method described above. The results of the maximum values of the height above the ground of the lines selected for the evaluation are summarized in FIG. 12B. According to the results of FIG. 12B, it can be concluded that:

i—Transgenic plants reach a height similar to wild plants towards the end of the life cycle.

II) Ubi::ZmRA1 plants show an increase in the number of leaves per plant. FIG. 12C documents the increase in the number of leaves per plant compared to wild plants.

To quantify the increase in the number of leaves, the number of leaves has been counted every 10 days up to 40 days after germination. According to FIG. 12C, it can be concluded that:

The plants of the present invention are characterized by having 2.35 times more leaves than wild plants. This trend is seen from the beginning of the life cycle and is accentuated towards 30 days after germination.

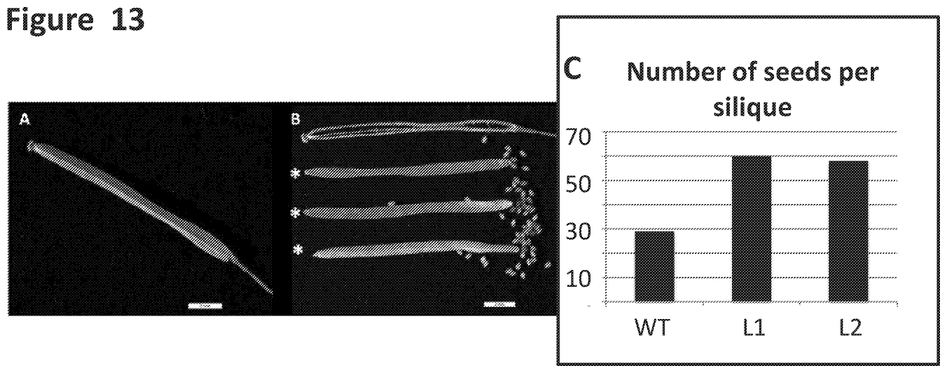

III) Ubi::ZmRA1 plants show an increase in the number of seeds per silique. FIG. 13 documents the increase in seed production. According to FIG. 13, it can be concluded that:

Transgenic plants showed a 200% increase in seed production per silique and a consequent increase in seed production per plant under normal growing conditions. This result is possibly due to the existence of an additional locule in the silique of transgenic plants compared to two locules in wild plants.

In summary, based on the evaluation of ZmRA1 transgenic plants, it can be concluded that the presence of the ZmRA1 transgene has a positive effect on the number of leaves and the production of seeds. These characteristics are suitable for food or forage production.

In addition, a nucleic acid encoding the zinc finger protein ZmRA1 can be used for plant breeding programs with a view to develop higher yielding plants.

The results presented indicate that the ZmRA1 technology can be used to obtain plants with improved agronomic characteristics.

Characterization of the Phenotypes of Transgenic Rice Ubi::ZinRA1 Plants.

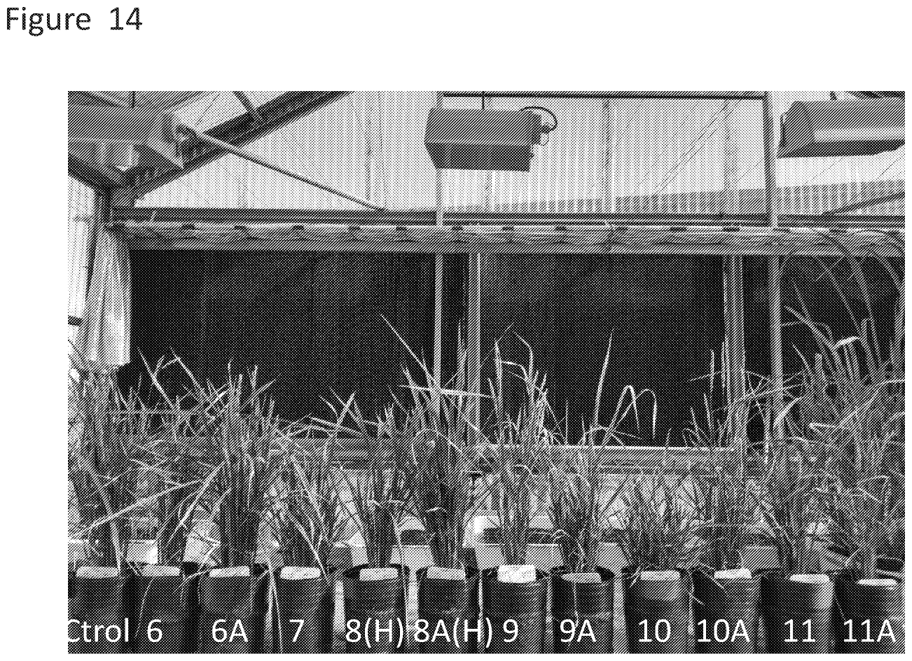

Eighteen independent T0 plants were obtained and their seeds harvested (T1). Six T1 events were selected for further analysis. Ten plants per T1 event were cultivated in the greenhouse (with 30° C./25° C. (day/night) and 16 h light/8 h dark at 50%-60% humidity with a light intensity of 20,000-25,000 lux) and their seeds harvested (T2). Stable homozygotes T2 plants were grown at greenhouse (with 30° C./25° C. (day/night) and 16 h light/8 h dark at 50%-60% humidity with a light intensity of 20,000-25,000 lux) and analyzed. Plants were irrigated 3 times a week and fertilized with with Basacote Plus 6M (5 grs/L) once. Examples of transgenic plants with low intermediate and height levels of expression are presented in FIG. 14.

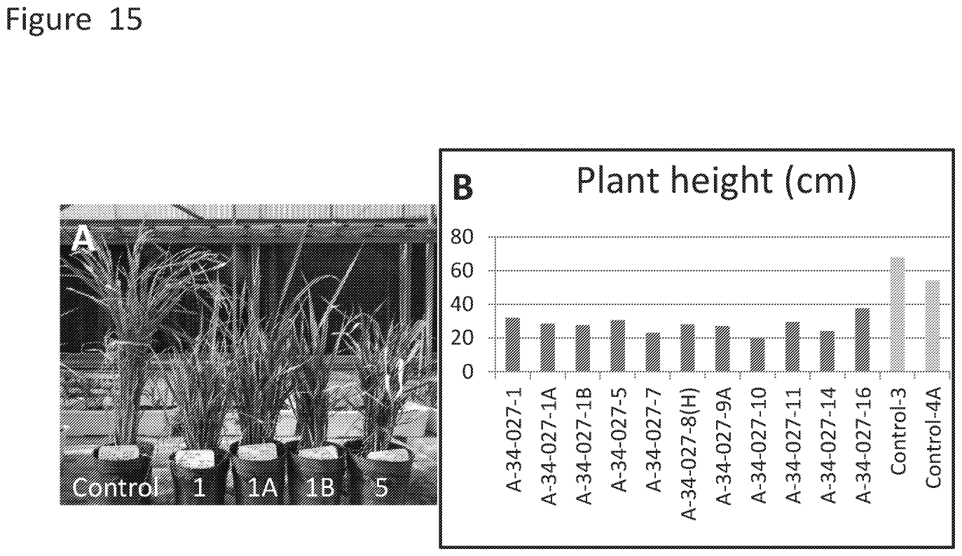

I) Ubi::ZmRA1 rice plants have a semi-dwarf phenotype. FIG. 15A documents the semi-dwarf phenotype of transgenic plants.

The height of the plant was calculated using photographs taken every month up to 150 days after germination. The height of the plant was determined following the method described above (the taller reproductive tiller was used as reference of the maximum height of a plant). The results of the maximum values of the plant height above the ground of the selected lines are summarized in FIG. 15B. According to the results of FIG. 15, it can be concluded that:

i—Transgenic rice plants present a semi-dwarf phenotype compared to control plants.

ii—The plants of the present invention are characterized by having strong stems resistant to overturning.

iii—Transgenic rice plants present an up-right phenotype compared to control.

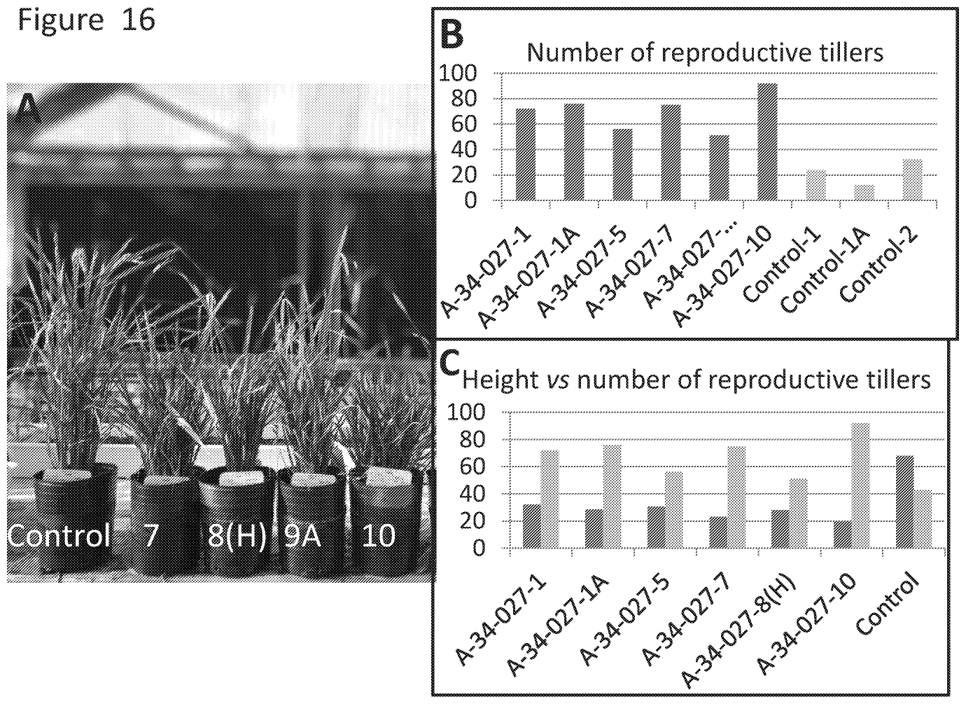

II) Ubi::ZmRA1 plants show an increase in the number reproductive tillers per plant. FIG. 16A documents the increase in the number of tillers per plant compared to control plants.

To quantify the increase in the number of reproductive tillers, the number of tillers with inflorescences was counted (FIG. 16B). Additionally, plant height was compared with number of reproductive tillers of transgenic plants and control plants (FIG. 16C). According to FIG. 16, it can be concluded that:

i—The plants of the present invention are characterized by having between 2 and 3 times more reproductive tillers than control plants.

ii—Given the inflorescences have similar yield compared to control plants, and increase in the number of reproductive tillers of transgenic plants represent an increase in yield per pot.

iii—The plants of the present invention are characterized by the a semi-dwarf and a high branching phenotypes in comparison to tall and less branching control plants.

In summary, based on the analysis of Ubi::ZmRA1 transgenic rice plants, it can be concluded that the presence of the ZmRA1 transgene has a positive effect on the size of the plant, as well as strength of the stems. These are desirable characteristic for any crop species since it prevents plant overturning events that considerably reduce production.

The up-right phenotype is a desirable characteristic for any species of cereal since it permits more plants per cultivated area.

Based on the analysis of ZmRA1 transgenic rice plants, it can be concluded that the presence of the ZmRA1 transgene has a positive effect on the number of reproductive tillers and the production of seeds. These characteristics are suitable for food or forage production.

In addition, a nucleic acid encoding the zinc finger protein ZmRA1 can be used for plant breeding programs with a view to developing higher yielding plants.

The results presented indicate that the ZmRA1 technology can be used to obtain plants with improved agronomic characteristics.

### BIBLIOGRAPHY

Ausubel F M, Brent R, Kingston R E, Moore D D, Seidman J G, Smith J A, Struhl K. 1983. Curr Protocol Mol Biol. John Wiley and Sons, New York.
Bailey T, Boden M, Buske F A, Frith M, Grant C E, Clementi L, Ren J, Li W W, Noble W S. 2009. MEME SUITE: tools for motif discovery and searching. Nucleic Acids Research 37:W202-W208.
Bimboim H C, Doly J. 1979. A rapid alkaline extraction procedure for screening recombinant plasmid DNA. Nucleic Acids Res 7: 1513-1523.
Brayer K J, Segal D J. 2008. Keep your fingers off my DNA: Protein-protein interactions mediated by C2H2 zinc finger domains. Cell Biochemistry and Biophysics 50: 111-131.
Cassani E, Landoni M, Pilu R. 2006. Characterization of the Rat maize gene involved in inflorescence architecture. Sex Plant Reproduction 19: 145-150.
Clough S J, Bent A F. 1998. Floral dip: a simplified method for Agrobacterium mediated transformation of Arabidopsis thaliana. The Plant Journal 16, 735-743.
Doyle J J, Doyle J L. 1990. A rapid total DNA preparation procedure for fresh plant tissue. Focus 12:13-15.
Englbrecht C C, Schoof H, Böhm S. 2004. Conservation, diversification and expansion of C2H2 zinc finger proteins in the Arabidopsis thaliana genome. BMC Genomics 5: 39.
Gallavotti A, Long J A, Stanfield S, Yang X, Jackson D, Vollbrecht E, Schmidt R J. 2010. The control of axillary meristem fate in the maize ramosa pathway. Development 137: 2849-2856.
Hanas J S, Hazuda D J, Bogenhagen D F, Y-h U F W, Wull C-W. 1983. Xenopus Transcription RNA Gene Factor A Requires Zinc for Binding to the 5 s. The Journal of Biological Chemistry 258: 14120-14125.
Huelsenbeck J P, Ronquist F. 2001. MRBAYES: Bayesian inference of phylogenetic trees. Bioinformatics 17: 754-755.
Isernia, C, Bucci E, Leone M, Zaccaro L, Di Lello P, Digilio G, Esposito S, Saviano M, Di Blasio B, Pedone C, Pedone P V, Fattorusso R. 2003. NMR Structure of the Single QALGGH Zinc Finger Domain from the Arabidopsis thaliana SUPERMAN Protein. Chembiochem 4: 171-180.
Katoh K, Misawa K, Kuma K, Miyata T. 2002. MAFFT: a novel method for rapid multiple sequence alignment based on fast Fourier transform. Nucleic Acids Research 30: 3059-3066.
Ke J, Ma H, Gu X, Thelen A, Brunzelle J S, Li J H, Xu E, Melcher K. 2015. Structural basis for recognition of diverse transcriptional repressors by the TOPLESS family of corepressors—Science Advances doi: 10.1126/sciadv.1500107.
Krishna S S, Majumdar I, Grishin N V. 2003. Structural classification of zinc fingers. Nucleic Acids Research 31: 532-550.
Laity J H, Lee B M, Wright P E. 2001. Zinc finger proteins: New insights into structural and functional diversity. Current Opinion in Structural Biology 11: 39-46.
Landoni M, Cassani E, Pilu R. 2007. Arabidopsis thaliana plants overexpressing Ramosal maize gene show an increase in organ size due to cell expansion. Sexual Plant Reproduction 20: 191-198.
Main M, Frame B, Wang K. 2015. Agrobacterium Protocols: Volume 1, Methods in Molecular. Biology, Wang K (ed.), vol 1223, DOI 10.1007/978-1-4939-1695-5 13, Springer Science+Business media New York, US.
Malgieri G, Palmieri M, Russo L, Fattorusso R, Pedone P V, Isernia C. 2015. The prokaryotic zinc-finger: Structure, function and comparison with the eukaryotic counterpart. The FEBS Journal 282: 4480-4496.
Mann D G J, LaFayette P R, Abercrombie L L, King Z R, Mazarei M, Halter M C, Poovaiah C R, Baxter H, Shen H, Dixon R A, Parrott W A, Stewart C N J R. 2012. Gateway-compatiblevectors for high-throughput gene functional analysis in switchgrass (Panicum virgatum L.) and other monocot species. Plant Biotechnoly Journal 10: 226-236.
Miller J, McLachlan A D, Klug A. 1985. Repetitive zinc-finger domains in the protein transcription factor IIIA from Xenopus oocytes. The EMBO Journal 4: 1609-1614.
Nylander J A A. 2004. MrModelTest 2.0. Program distributed by the author. Evolutionary Biology Centre, Uppsala University. (http://www.ebc.uu.se/systzoo/staff/nylander.html).
Ohta M, Matsui K, Hiratsu K, Shinshi H, Ohme-Takagia M. 2001. Repression domains of Class II ERF transcriptional repressors share an essential motif for active repression. The Plant Cell 13: 1959-1968.
Rambaut A, Drummond A J. 2007. Tracer v1.5. http://beast.bio.ed.ac.uk/Tracer.
Tamura K, Stecher G, Peterson D, Filipski A, Kumar S. 2013. MEGA6: Molecular Evolutionary Genetics Analysis Version 6.0. Molecular Biology and Evolution 30: 2725-2729.
Sakai H, Medrano L J, Meyerowitz E M. 1995. Role of SUPERMAN in maintaining Arabidopsis floral whorl boundaries. Nature 378: 199-203.
Sakamoto H, Araki T, Meshi T, Iwabuchi M. 2000. Expression of a subset of the Arabidopsis Cys2/His2-type zinc-finger protein gene family under water stress. Gene 248: 23-32.
Sambrook J, Fritsc, E F, Maniatis T. 1989. Molecular cloning: a laboratory manual. Seconf edition. Colf Spring Abstract.
Soreng R J, Peterson P M, Romaschenko K, Davidse G, Zuloaga F O, Judziewicz E J, Filgueiras T S, Davis J I, Morrone O. 2015. A worldwide phylogenetic classification of the Poaceae (Gramineae). Journal of Sytematics Evolution 53: 117-137.
Takatsuji H, Mori M, Benfey P N, Ren L, Chua N H. 1992. Characterization of a zinc finger DNA-binding protein expressed specifically in Petunia petals and seedlings. The EMBO Journal 11: 241-249.
Takatsuji H. 1998. Zinc-finger transcription factors in plants. Cellular and Molecular Life Sciences 54: 582-596.
Troll W. 1964. Die Infloreszenzen: Typologie and Stellung im Aufbau des Vegetationskopers. Fisher, Stuttart.
Tupler R, Perini G, Green M R. 2001. Expressing the human genome. Nature 409: 832-833.
Vollbrecht E, Springer P S, Goh L, Buckler V I E S, Martienssen R. 2005. Architecture of floral branch systems in maize and related grasses. Nature 436: 1119-1126.
Yang X. 2001. Study of RAMOSA1 function during maize inflorescence development. Graduate Theses and Dissertations. Iowa State University of Science and Technology, Plant Biology Department, Ames, Iowa, USA.

1.-31. (canceled)

32. A method to improve agronomic characteristics of plants comprising genetically transforming a plant with a nucleic acid sequence encoding a RAMOSA1 transcription factor, wherein the plant is selected from the group consisting of monocotyledonous grasses of BOP clade, non-grass monocotyledonous and dicotiledoneous.

33. The method of claim 32, wherein said nucleic acid sequence encoding the RAMOSA1 transcription factor from plants is of PACMAD clade.

34. The method of claim 32, wherein said nucleic acid sequence encoding the RAMOSA1 transcription factor is from plants of the genus Setaria, Cenchrus or Zea.

35. The method of claim 34 wherein the species is Setaria viridis, Cenchrus equinatus or Zea mays.

36. The method of claim 32, wherein said nucleic acid sequence encoding the RAMOSA1 transcription factor comprises a sequence selected from the group consisting of SEQ ID NO: 1 SEQ ID NO: 2 AND SEQ ID NO: 3.

37. The method of claim 32 wherein said nucleic acid sequence encoding the RAMOSA1 transcription factor is overexpressed with a plant or seed promoter.

38. The method of claim 32 wherein the method confers an improvement selected from the group consisting of: increases at least 30% seed production; at least double the biomass; extend at least 100% the life of the transformed plant; and a combination thereof.

39. An isolated DNA comprising a sequence with at least 95% homology to nucleic acid sequence selected from the group consisting of: SEQ ID NO: 1, SEQ ID NO: 2 AND SEQ ID NO: 3.

40. The isolated DNA of claim 39, wherein sequence comprises at least 99% homology to any of the sequence selected from the group consisting of: SEQ ID NO: 1, SEQ ID NO: 2 AND SEQ ID NO: 3.

41. The isolated DNA of claim 39, wherein the DNA sequence is selected from the group SEQ ID NO: 1, SEQ ID NO: 2 AND SEQ ID NO: 3.

42. The isolated DNA sequence of claim 39, wherein said isolated DNA sequence is cDNA.

43. The isolated DNA sequence of claim 39, further comprising a promoter for overexpression of said DNA sequence selected from the group consisting of: promoters of the actin, ubiquitin, ZmUbi1100, pEMU, MAS genes, H4 histone of maize, rice, Panicum virgatum, Setaria; promoter of peanut chlorotic caulimovirus (PCISV); 35S cauliflower mosaic virus promoter (CaMV); the complete promoter of tabacco mosaic virus (FMV); the ALSO gene promoter from Brassica napus; promoters of Agrobacterium genes; and promoters of Setaria viridis, Cenchrus quinatus and Zea maiz.

44. The isolated DNA according to claim 39, wherein said DNA comprises an expression control sequence, said nucleic acid sequence and optionally, a sequence of termination of transcription.

45. The method according to claim 32, wherein the improvement is to increase: biomass, root growth, seed production, or the life of a plant comprising introducing a nucleic acid of a sequence selected from the group consisting of SEQ ID NO: 1, SEQ ID NO: 2 and SEQ ID NO: 3 in the plant.

46. The method to improve agronomic characteristics of transgenic plants compared to wild plants according to claim 32, wherein said characteristics are selected from increased biomass, increased root growth, increased seed production and increased plant life, wherein the method comprises:
a. introducing into a plant or a plant cell a nucleic acid of a sequence selected of the group consisting of: SEQ ID. NO: 1, SEQ ID. NO: 2 and SEQ ID. NO: 3;
b. cultivating the plant or plant cell under conditions that promote its growth.

47. The method to improve agronomic characteristics of transgenic plants compared to wild plants, according to claim 32, wherein said agronomic characteristics are selected from the group consisting of: increasing its biomass at least 50%, at least 30% the production of seeds, and extends the life of the plant 100%, and wherein the method further comprises:
a. introducing into a plant or a plant cell a nucleic acid of a sequence selected of the group consisting of: SEQ ID NO: 1, SEQ ID NO: 2 and SEQ ID NO: 3;
b. cultivating the plant or plant cell under conditions that promote its growth.

48. A transgenic grass-monocotyledonous, non-grass monocotyledonous or dicotyledonous plant comprising a nucleic acid sequence encoding for RAMOSA1 transcription factor.

49. The transgenic grass-monocotyledonous, non-grass monocotyledonous or dicotyledonous plant of claim 48, wherein said nucleic acid sequence is selected of the group consisting of: SEQ ID NO: 1, SEQ ID NO: 2 and SEQ ID NO: 3.

50. The transgenic grass-monocotyledonous, non-grass monocotyledonous or dicotyledonous plant of claim 48 wherein, in comparison with the native grass-monocotyledonous, non-grass monocotyledonous or dicotyledonous plant, has increased its biomass, seed production and life cycle.


SEQUENCE LISTING (6 sequences)

SEQ ID NO: 1 - DNA, 528 bp, Zea Mays
atggagggagaagatgacggcgcccaaatgaaactgcagcaacaacaacagtcgccttgcagtgacaacttgagcttgtccgccgcctcctcatggctgccgccacaggtaaggtcgtcgtcgtcgtcgtcgtcgtacacctgcgggtattgcaagaaggagttcagatcagcacaagggctgggaggccacatgaacatccacaggctggacagggccagactgatccaccaacagtacacttcacaccgtattgctgctccccatccaaaccctaatcctagttgcacatcagttcttgaccttgagctcagcttgtcgtcgctgctagcgcatggtgctgccagcagcgacggaggcttgtctgttccagtggcaaagctggcgggcaaccgtttctcctccgcatcgctccccacgaccaaggacgtcgaggggaagaacttagagttgaggataggagcgtgcagtcatggcgatggcgcggaagagcgtctggatcttcagcttagactgggctactactga

SEQ ID NO: 2 - DNA, 522 bp, Setaria Viridis
atggagagagatgatggctacaccaagcttcagcagctaccttgcagcgacaacttcagcttggccgcctcctcttcatggccgtcgccacagataaggtcgtcgtcctccgcgacgtcccacatctgcgggtattgcaagagggagttcaggtcagcacaagggctgggaggtcacatgaacgtccacaggttggacagggccaggctgatccaccaccagtgctcctcacaccaccatctagctcttgctgctccccctccaaaccctaaccctagtcgcgcagttgttgaactcatgagctcaggttgtcgcgcgcacggtgctgccagcgatggaggctcggctgtgccgccagcggcaaagccgggcgtctgccgtttctccttggcatcgtcgtcgtccgccttgacaaaggacatcgaggcgatcaagaacttggagttgaggatgggagcgtgcagccatggcgacgacgcggaagagcgtctggatctagagctaagacttggccactcctga

SEQ ID NO: 3 - DNA, 546 bp, Cenchrus equinatus
atggagagagatgatggctacaccaagctgcagcagctaacttgcagcgacaatttcagcttggctgcctcctcctcatggccacagtcacaaacaaggtcgccatcctccacgacatcccacgtctgcgcgtattgcaagagggagttcaggtcagcacaagggatgggaggtcacatgaacgtccacaggttggacagggccaggctgatccaccaccagtgctcctcaccacaccaccatcatctagctcttgcagctccccctccaaaccctaaccctagtcgcacagctgctgaactcttgggctccggcggtcacgctcgcgcgcgcggtgctgctgccagtgatggaggctcagctgtgccgccgccagcggcaaagccgccgggtgtctgccgccgtttttccttggcatcgtcgccgtccgcctcgacgaaagacatggaggtgatgaggaccttggagttgaggatggggacgtgcagccatggtgacggcgcggaagagcgtctagatcttgagcttagacttggctactcctga

SEQ ID NO: 4 - protein, 175 aa, Zea mays
MEGEDDGAQMKLQQQQQSPCSDNLSLSAASSWLPPQVRSSSSSSSYTCGYCKKEFRSAQGLGGHMNIHRLDRARLIHQQYTSHRIAAPHPNPNPSCTSVLDLELSLSSLLAHGAASSDGGLSVPVAKLAGNRFSSASLPTTKDVEGKNLELRIGACSHGDGAEERLDLQLRLGYY

SEQ ID NO: 5 - protein, 173 aa, Setaria viridis
MERDDGYTKLQQLPCSDNFSLAASSSWPSPQIRSSSSATSHICGYCKREFRSAQGLGGHMNVHRLDRARLIHHQCSSHHHLALAAPPPNPNPSRAVVELMSSGCRAHGAASDGGSAVPPAAKPGVCRFSLASSSSALTKDIEAIKNLELRMGACSHGDDAEERLDLELRLGHS

SEQ ID NO: 6 - protein, 181 aa, Cenchrus equinatus
MERDDGYTKLQQLTCSDNFSLAASSSWPQSQTRSPSSTTSHVCAYCKREFRSAQGMGGHMNVHRLDRARLIHHQCSSPHHHHLALAAPPPNPNPSRTAAELLGSGGHARARGAAASDGGSAVPPPAAKPPGVCRRFSLASSPSASTKDMEVMRTLELRMGTCSHGDGAEERLDLELRLGYS